(19) United States
(12) Patent Application Publication
(10) Pub. No.: US 2015/0230744 A1
(43) Pub. Date: Aug. 20, 2015

(54) METHOD AND SYSTEM FOR ASSESSING A STIMULUS PROPERTY PERCEIVED BY A SUBJECT

(71) Applicant: VALORISATION-RECHERCHE, LIMITED PARTNERSHIP, MONTREAL (CA)

(72) Inventors: Jocelyn Faubert, Montreal (CA); Claudine Habak, Saint-Laurent (CA); Rafael Doti, Montreal (CA); Jesus-Eduardo Lugo-Arce, Laval (CA)

(21) Appl. No.: 14/420,536

(22) PCT Filed: Aug. 9, 2013
(86) PCT No.: PCT/CA2013/000712

§ 371 Date: Feb. 9, 2015

Related U.S. Application Data
(60) Provisional application No. 61/682,022, filed on Aug. 10, 2012.

(51) Int. Cl. A61B 5/1455; A61B 3/032; A61B 5/0484

(52) U.S. Cl. CPC A61B 5/14553; A61B 5/04842; A61B 5/04845; A61B 5/04847; A61B 3/032

(57) ABSTRACT

A method for assessing a subject perception of a stimulus property, comprising: applying to a subject at least a first sensory stimulus having a first value for the stimulus property and a second sensory stimulus having a second value for the stimulus property different from the first value; requesting the subject to identify a characteristic of the first and second sensory stimuli during the application thereof, the characteristic being unrelated to the stimulus property to be assessed, thereby focusing an attention of the subject on a decision making task; measuring an activity of at least one brain region of the subject during the application of the first and second sensorial stimuli and the identification of the characteristic thereof, thereby obtaining a brain activity measurement; and determining a difference of stimulus property perceived by the subject between the first and second sensory stimuli from the brain activity measurement.

### CROSS-REFERENCE TO RELATED APPLICATIONS

This application claims priority of US Provisional Patent Application having Ser. No. 61/682,022, which was filed on Aug. 10, 2012 and is entitled “Method and system for assessing a stimulus property perceived by a subject”, the specification of which is hereby incorporated by reference.

### TECHNICAL FIELD

The present invention relates to the field of methods and systems for assessing a stimulus property perceived by subjects.

### BACKGROUND

Determining how a sensory stimulus is perceived by a subject may be of great importance for identifying pathologies. For example, determining whether a subject sees an image as clear or not may help a clinician or an optometrist to assess any visual perception pathologies.

Some of the existing methods for assessing how a subject perceives a sensory stimulus rely on the subject himself, i.e. the subject is asked to evaluate the clarity of the stimulus he perceives. Such methods are therefore subjective and may not be precise enough under certain circumstances. For example, a subject may not be able to differentiate two stimuli of which the difference of clarity or intensity is low.

Therefore, there is a need for an improved method and system for assessing a subject perception of sensory clarity.

### SUMMARY

In accordance with a first broad aspect, there is provided a method for assessing a subject perception of a stimulus property, comprising: applying to a subject at least a first sensory stimulus having a first value for the stimulus property and a second sensory stimulus having a second value for the stimulus property, the second value being different from the first value; requesting the subject to identify a characteristic of the first and second sensory stimuli during the application thereof, the characteristic being unrelated to the stimulus property to be assessed, thereby focusing an attention of the subject on a decision making task; measuring an activity of at least one brain region of the subject during the application of the first and second sensorial stimuli and the identification of the characteristic thereof, thereby obtaining a brain activity measurement; and determining a difference of stimulus property perceived by the subject between the first and second sensory stimuli from the brain activity measurement, thereby characterizing the subject perception of the stimulus property.

In one embodiment, the stimulus property comprises a stimulus clarity.

In another embodiment, the stimulus property comprises one of a contrast and a noise.

In one embodiment, the first sensory stimulus and the second sensory stimulus each comprise a visual stimulus.

In another embodiment, the first sensory stimulus and the second sensory stimulus each comprise one of an auditory stimulus and a tactile stimulus.

In a further embodiment, the first sensory stimulus and the second sensory stimulus each comprise one of an olfactory stimulus and a gustatory stimulus.

In one embodiment, the requesting step comprises requesting the subject to identify the characteristic of the first and second sensory stimuli one of verbally, mentally, and via an input device.

In one embodiment, the requesting step comprises requesting the subject to assign a given category to which the characteristic belongs, the given category being chosen amongst predetermined categories.

In one embodiment, the measuring step comprises measuring the activity in a corresponding brain region responsible for integrating a sensory information related to the first and second sensory stimuli.

In one embodiment, the measuring step comprises measuring the activity in at least one region of a frontal cortex facing a forehead of the subject.

In one embodiment, the measuring step comprises measuring at least one of an electrical activity, a blood flow, a blood oxygenation, and a temperature.

In one embodiment, the applying step comprises displaying a first image having the first value for the stimulus property and a second image having the second value for the stimulus property.

In one embodiment, the determining step comprises determining a relative entropy between the first and second sensory stimuli and characterizing the subject perception of the stimulus property using the relative entropy.

In accordance with a second broad aspect, there is provided a system for assessing a subject perception of a sensory property, comprising: a stimulus generator for applying to a subject at least a first sensory stimulus having a first value for the stimulus property and a second sensory stimulus having a second value for the stimulus property different from the first value, the first and second sensory stimuli each comprising a characteristic to be identified by the subject during the application thereof in order to focus an attention of the subject on a decision making task, the characteristic being unrelated to the stimulus property to be assessed; a cerebral activity sensing unit for measuring an activity of at least one brain region of the subject during the application of the first and second sensorial stimuli and the identification of the characteristic thereof in order to obtain a brain activity measurement; and a clarity perception determining unit for determining a difference of stimulus property perceived by the subject between the first and second sensory stimuli from the brain activity measurement in order to characterize the subject perception of the stimulus property.

In one embodiment, the stimulus generator is adapted to display a first image having a first value for a visual clarity and a second image having a second value for the visual clarity, the first and second values for the visual clarity being set by adjusting a frequency amplitude spectrum of a given image.

In one embodiment, the cerebral activity sensing unit comprises one of an electroencephalography device, a functional near infrared spectroscopy device, and a functional magnetic resonance imaging device.

In accordance with a third broad aspect, there is provided a computer-implemented method for assessing a subject perception of a stimulus property, comprising: generating and transmitting to a stimulus generator a command indicative of at least a first sensory stimulus and a second sensory stimulus to be applied to the subject by the stimulus generator, the first sensory stimulus having a first value for the stimulus property and the second sensory stimulus having a second value for the stimulus property different from the first value, the first and second sensory stimuli each comprising a characteristic to be identified by the subject during the application thereof in order to focus an attention of the subject on a decision making task, the characteristic being unrelated to the stimulus property to be assessed; receiving, from a cerebral activity sensing unit, a measurement of an activity of at least one brain region of the subject during the application of the first and second sensorial stimuli and the identification of the characteristic thereof; and determining a difference of stimulus property perceived by the subject between the first and second sensory stimuli from the received measurement; and outputting the difference of stimulus property perceived by the subject.

In accordance with another broad aspect, there is provided a method for identifying an adequate corrective lens for a subject, comprising: for each one a first and a second corrective lens worn by the subject: displaying to the subject at least a first image having a first degree of clarity and a second image having a second degree of clarity different from the first degree of clarity; requesting the subject to identify a characteristic of the first and second images during the displaying thereof, the characteristic being unrelated to the clarity of the first and second images, thereby focusing an attention of the subject on a decision making task; measuring an activity of at least one brain region of the subject during the displaying of the first and second images and the identification of the characteristic thereof, thereby obtaining a brain activity measurement; and determining a difference of clarity perceived by the subject between the first and second images from the brain activity measurement, thereby obtaining the subject's perception of sensory clarity; and identifying the adequate corrective lens as being the one of the first and second corrective lenses having the greatest difference of clarity perceived by the subject.

In accordance with a further broad aspect, there is provided a system for identifying an adequate corrective lens for a subject, comprising: an image generator for generating at least a first image having a first degree of clarity and a second image having a second degree of clarity different from the first degree of clarity, the first and second images each comprising a characteristic to be identified by the subject during the displaying thereof in order to focus an attention of the subject on a decision making task, the characteristic being unrelated to the clarity of the first and second images; a display unit for displaying to the subject the first and second images received from the image generator; a cerebral activity sensing unit for measuring an activity of at least one brain region of the subject during the displaying of the first and second images and the identification of the characteristic thereof in order to obtain a brain activity measurement; and an adequate lens determining unit adapted to, for each one a first and a second corrective lens iteratively worn by the subject: determine a difference of clarity perceived by the subject between the first and second images from the brain activity measurement, thereby obtaining the subject's perception of sensory clarity; and output the difference of clarity perceived by the subject for each one of the first and second corrective lens, the adequate corrective lens being identified as being the one of the first and second corrective lenses having the greatest difference of clarity perceived by the subject.

In accordance with still another embodiment, there is provided a computer-implemented method for identifying an adequate corrective lens for a subject, comprising: transmitting to a display unit at least a first image having a first degree of clarity and a second image having a second degree of clarity different from the first degree of clarity, the first and second images each comprising a characteristic to be identified by the subject during the displaying thereof in order to focus an attention of the subject on a decision making task, the characteristic being unrelated to the clarity of the first and second images; receiving, from a cerebral activity sensing unit, a measurement of an activity of at least one brain region of the subject during the displaying of the first and second images and the identification of the characteristic; and for each one a first and a second corrective lens iteratively worn by the subject: determining a difference of clarity perceived by the subject between the first and second images from the brain activity measurement, thereby obtaining the subject perception of sensory clarity; and outputting the difference of clarity perceived by the subject for each one of the first and second corrective lens, the adequate corrective lens being identified as being the one of the first and second corrective lenses having the greatest difference of clarity perceived by the subject.

### BRIEF DESCRIPTION OF THE DRAWINGS

Further features and advantages of the present invention will become apparent from the following detailed description, taken in combination with the appended drawings, in which:

FIG. 1 is a flow chart of a method for characterizing sensory clarity perceived by a subject, in accordance with an embodiment;

FIG. 2 is a block diagram of a system for characterizing sensory clarity perceived by a subject, in accordance with an embodiment;

FIG. 3 illustrates a helmet for sensing a frontal cortex activity, in accordance with an embodiment;

FIG. 4 illustrates two frontal cortex regions that are sensed by the helmet of FIG. 3, in accordance with an embodiment;

FIG. 5a illustrates an exemplary time series with amplitude plotted as a function of frequency for an original auditory stimulus;

FIG. 5b illustrates an exemplary power spectrum distribution of the original stimulus of FIG. 5a;

FIG. 5c illustrates an exemplary power spectrum distribution for a blurred auditory stimulus, in which power is plotted as a function of frequency in Hz;

FIG. 5d illustrates an exemplary power spectrum distribution for a sharpened auditory stimulus, in which power is plotted as a function of frequency in Hz;

FIG. 6a illustrates an exemplary time series with amplitude plotted as a function of frequency for an original tactile stimulus;

FIG. 6b illustrates an exemplary power spectrum distribution of the original stimulus of FIG. 6a;

FIG. 6c illustrates an exemplary power spectrum distribution for a blurred tactile stimulus, in which power is plotted as a function of frequency in Hz;

FIG. 6d illustrates an exemplary power spectrum power distribution for a sharpened tactile stimulus, in which power is plotted as a function of frequency in Hz;

FIGS. 7a and 7b illustrates examples of Fourier transform of infrared spectra showing peaks for toluene and benzene, respectively;

FIG. 8 illustrates one example of a Fourier transform of infrared spectra showing the peaks of glucose (sugar) and sodium chloride (salt) for a gustatory stimulus;

FIG. 9 illustrates seven images each having a different level of clarity, in accordance with an embodiment;

FIG. 10 illustrates a temporal order of display for the images of FIG. 9, in accordance with an embodiment;

FIG. 11a illustrates an exemplary blood oxygenation in time for one channel during application of a plurality of different stimuli;

FIG. 11b illustrates an exemplary location in time of the stimuli of FIG. 11a;

FIG. 11c illustrates an exemplary blood oxygenation in time corresponding to a given one of the stimuli of FIG. 11a;

FIG. 11d illustrates the blood oxygenation for the given stimuli of FIG. 11d as a single time sequence;

FIG. 12 illustrates an exemplary average blood oxygenation value as a function of channels for three exemplary conditions;

FIG. 13a illustrates an exemplary blood oxygenation averaged over time and over channels;

FIG. 13b illustrates an exemplary graph of oxygenation change as a function of a variation of image clarity level for different subjects;

FIG. 14 is an exemplary graph illustrating an average blood oxygenation per channel;

FIG. 15 is an exemplary graph presenting a difference of entropy for seven different visual stimuli;

FIG. 16 is an exemplary graph illustrating a difference of entropy for a subject, with and without corrective lenses;

FIG. 17 illustrates a procedure for psychophysical adaptation condition, in accordance with an embodiment;

FIG. 18 illustrates an optode array placement for occipito-temporal cerebral activity measurement, in accordance with an embodiment;

FIG. 19 illustrates an exemplary block design during which participants categorized images as mammals or birds;

FIG. 20a is an exemplary graph of point of subjective equality as a function of observers resulting from a behavioral analysis;

FIG. 20b is an exemplary graph of magnitude of adaptation shift after adaptation to blur (blue squares) and sharp (black triangles) as a function of observers, resulting from a behavioral analysis;

FIGS. 21a-21j are exemplary graphs of individual data with proportion “sharpened” responses as a function of image slope for different observers, resulting from a behavioral analysis; and

FIG. 22 illustrates an exemplary event-related design during which participants categorized images as cats or dogs.

FIGS. 23a-c each illustrate an exemplary graph of a relative entropy as a function of an image clarity level for a repetition of a same condition over 5 sessions, for a respective subject;

FIG. 24a illustrates an exemplary graph of a relative entropy as a function of an image clarity level for multiple runs over a single session, the relative entropy being determined from oxy-hemoglobin;

FIG. 24b illustrates an exemplary graph of a relative entropy as a function of an image clarity level for multiple runs over a single session, the relative entropy being determined from total-hemoglobin;

FIGS. 25a-i each present an image of a red panda having a respective clarity level;

FIG. 26a-c each illustrate, for a respective subject, an exemplary graph of a relative entropy as a function of the image clarity level when the respective subject is presented with the images of FIGS. 25a-i;

FIGS. 27a-d each illustrate, for a respective subject, an exemplary graph of a relative entropy as a function of the image clarity level when the respective subject is presented with images presenting nine different levels of clarity including ±0.025;

FIGS. 28a-b each illustrate, for a respective subject, an exemplary graph of a relative integral of the entropy's power spectrum density as a function of the image clarity level, with the levels being rank-ordered by entropy;

FIGS. 28c-d each illustrate, for a respective subject, an exemplary graph of an absolute integral of an entropy power spectrum density for an original image only;

FIG. 29a illustrates an exemplary graph of an absolute integral of power spectrum density as a function of an image clarity level;

FIG. 29b illustrates an exemplary graph of the slope of the linear fit estimated in FIG. 29a;

FIGS. 30a-c each illustrate, for a respective subject, an exemplary graph of a slope of the absolute integral of power spectrum density as a function of the image clarity level for a first prescription shift set;

FIGS. 31a-c each illustrate, for a respective subject, an exemplary graph of a slope of the absolute integral of power spectrum density as a function of the image clarity level for a second prescription shift set;

FIGS. 32a-d each illustrate an exemplary graph of a slope of the absolute integral of power spectrum density as a function of the image clarity level for a same participant and for different days;

FIGS. 33a-b each illustrate en exemplary graph of a relative entropy as a function of an image clarity, the relative entropy being determined from the activity of an occipital cortex and a frontal cortex, respectively;

FIG. 34 illustrates an exemplary graph of a diopter as a function of an image clarity level

It will be noted that throughout the appended drawings, like features are identified by like reference numerals.

### DETAILED DESCRIPTION

There is described a method and system for assessing the perception of a stimulus property or characteristic of a subject in response to a sensory stimulus applied to the subject. The method and system allows for determining a difference of perceived stimulus property between two different stimuli while not relying on any stimulus property characterization performed by the subject. The method comprises a step of measuring the subject's cerebral activity while applying two different stimuli having a different value for a same stimulus property to the subject and requesting the subject to maintain his attention of a decision-making task which is unrelated to the stimulus property. The difference of perceived stimulus property between the two stimuli is determined from the cerebral activity measurement.

For example, visual clarity is variable, as both optical and environmental factors induce blur on the retinal image, but most individuals perceive our world in-focus. Clinically, some individuals adapt to ocular correction better than others, and therefore have varying success rates from correction. This tolerance to blur in perceived image quality/clarity depends not only on optical factors, but also on individual experiences and neural function. When faced with change, brain's neural systems are capable of adapting, so that the subject's visual system maintains an operating range that is suited to our environment. The perception of image clarity, therefore, relies on the individual state of adaptation and is variable.

Therefore, measuring cerebral activity may help in assessing the clarity perceived by individuals in a substantially objective manner. By maintaining substantially constant the activity of a subject's brain except the cerebral activity related to the sensory stimuli, one can determine that the variation of cerebral activity between different stimuli is substantially related to the difference of clarity between the stimuli. In the present method and system, the cerebral activity caused by the functions unrelated to the sensory stimuli is maintained substantially constant by focusing the subject's attention on a decision-making task.

The present method and system may be used for assessing a perceived stimulus property of any type of sensory stimulus, i.e. a visual, auditory, tactile, olfactory, or gustatory stimulus. In one embodiment, in order to determine the perceived stimulus property of a given sensory stimulus, the cerebral activity related to the given sensory stimulus is measured in the corresponding region responsible for the integration of the given sensory information, i.e “lower-level” modality-specific brain regions. For example, the cerebral activity related to a visual stimulus is measured in at least one region of the visual cortex, the cerebral activity related to an auditory stimulus is measured in at least one region of the auditory cortex, etc.

In another embodiment, in order to determine the perceived stimulus property of a given sensory stimulus, the cerebral activity related to the given sensory stimulus is measured in at least one region of the frontal cortex facing the forehead of the subject. At least some of the “lower-level” modality-specific brain regions are covered with hair, which may decrease the quality of sensing signals. For example, probes such as electroencephalography (EEG) sensors may be positioned on top of the individual's head for sensing the cerebral activity of a given sensory cortex. However, hair is located between the EEG sensors and the sensory cortex, and may affect the quality of the sensing signals received from the cortex.

The inventors have unexpectedly discovered that it is possible to obtain the brain activity of a “lower-level” brain center, i.e. a sensory cortex, by measuring the cerebral activity of a “higher-level” brain center such as the frontal cortex, and therefore determine the perceived stimulus property of the sensory stimulus from the frontal cortex activity. The frontal cortex executive functions involve the ability to recognize future consequences resulting from current actions, to choose between good and bad actions (or better and best), override and suppress unacceptable social responses, and determine similarities and differences between things or events. The inventors have discovered that when, a subject is asked to make a decision while a sensory stimulus is applied to the subject, the sensory information is provided from the sensory cortex to the frontal cortex along with the decision-making information, so that the activity of the frontal cortex is not only related to the decision-making but also to the clarity perception of the sensory stimulus. The inventors have discovered that when a subject is asked to make a decision while a sensory stimulus is applied to the subject, the sensory information is provided from the sensory cortex to the occipital cortex along with the decision-making information, so that the activity of the occipital cortex is not only related to the decision-making but also to the clarity perception of the sensory stimulus. Therefore, by measuring the frontal cortex activity, the perceived stimulus property of a sensory stimulus may be determined, and since the forehead is usually provided with substantially no hair, the quality of the sensing signals is not affected by the hair.

In a further embodiment, in order to determine the perceived stimulus property of a given sensory stimulus, the cerebral activity related to the given sensory stimulus is measured in at least one region located at the frontier of the occipital cortex, the temporal cortex, and the parietal cortex.

In the following, a method and system for assessing the perception of clarity of a subject in response to a sensory stimulus based on the above comments are described. The sensory stimulus may be any type of natural stimulus such as a visual, auditory, tactile, olfactory, or gustatory stimulus.

It should be understood that the present method and system may be adapted to measure stimulus property other than the clarity.

A particular application of the present method and system for determining an adequate lens for a subject is further described below.

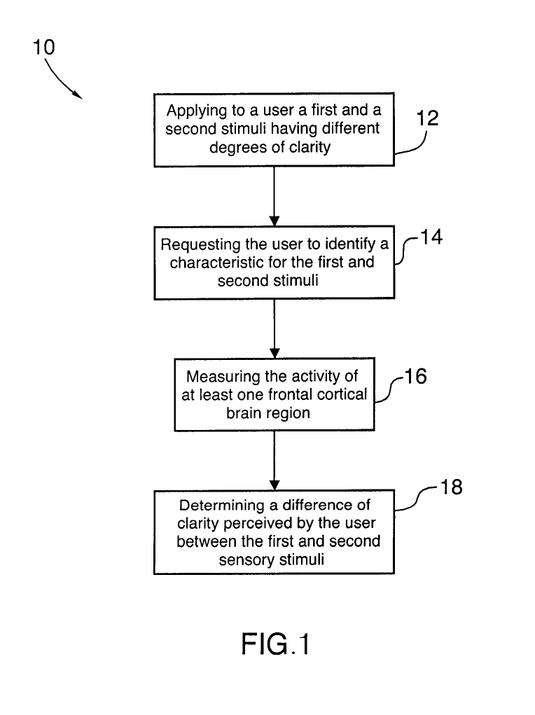

FIG. 1 illustrates one exemplary embodiment of a method 10 for determining the stimulus clarity perceived by a subject. At step 12, at least two natural stimuli are generated and applied to the subject. The two stimuli are of a same kind, i.e. they are both visual, auditory, tactile, olfactory, or gustatory stimuli, but present a different clarity value, i.e. a different degree of clarity. Furthermore, each stimulus comprises a characteristic to be identified by the subject, which is not related to the clarity of the stimuli.

At step 14, the subject is requested to identify the characteristic for each stimulus while the stimuli are applied to the subject. The purpose of the identification of the characteristic is to have the subject focus his attention on the stimulus and make a decision while each stimulus is applied to him.

In one embodiment, the subject is requested to verbally identify the characteristic of the stimuli. In another embodiment, the subject identifies the characteristic of the stimuli via a user input device. Therefore, during the application of each stimulus, the user provides his answer, i.e. the identification of the characteristic, either verbally or via an input device.

In a further embodiment, the subject is requested to mentally identify the characteristic while not providing his answer either verbally or via an input device.

In one embodiment, the characteristic to be identified is constant through the stimuli. For example, the subject may be requested to choose between a predetermined number of options for identifying the characteristic of the stimuli. In one example, the characteristic to be identified by the subject comprises a category to which the stimulus belongs. For example, two categories may be presented to the subject who is asked to identify the category to which each stimulus belongs.

For example, in the case where visual stimuli are applied to the subject and each visual stimulus comprises an image of a cat or a dog displayed to the subject, the characteristic of the image to be identified may be the cat/dog category to which the image belongs. By applying stimuli comprising a same characteristic to be identified, the activity of the “higher-level” brain centers related to the decision making, such as the frontal cortex is maintained substantially constant during the application of the method 10.

In one embodiment, the identification of the characteristic requires a substantially low activity from the “higher-level” brain centers. For example, the identification of the stimulus characteristic may consist in choosing between two elements such as “dog”/“cat”, “triangle/circle”, etc, as described above with respect to a visual stimulus. In one embodiment, the percentage of right answers to the identification of the characteristic is set to a predetermined value for ensuring a low activity for the “higher-level” brain centers. In this case, the characteristic to be identified is chosen so that an average subject would obtain the predetermined percentage of right answers while identifying the characteristic.

In one embodiment, the predetermined percentage of right answers is set to a minimum of 80%. In this case, the characteristic to be identified by the subject is chosen so that the subject is able to correctly identify the characteristic for a minimum of 80% of the stimuli. In another embodiment, the predetermined percentage of right answers is at least equal to 90%. In this case, the characteristic to be identified by the subject is chosen so that the subject is able to correctly identify the characteristic for 90% of the stimuli. This would ensure a substantially stable activity for the “higher-level” brain centers.

At step 16, the activity of at least one brain region of the subject is measured during the application of the stimuli to the subject and the identification of the characteristic by the subject. It should be understood that any adequate method and system for measuring cerebral activity may be used.

In one embodiment, the step 16 comprises measuring the cerebral activity of the sensory cortex corresponding to the sensory stimulus of which the perceived clarity is to be characterized. For example, the cerebral activity of at least one region of the visual cortex is measured when the visual perceived clarity is to be assessed. In another example, the cerebral activity of at least one region of the tactile cortex is measured in order to characterize the perceived tactile clarity. In another embodiment, the step 16 comprises measuring the cerebral activity of at least one region of the frontal cortex facing the forehead of the subject.

In one embodiment, the cerebral activity is measured by measuring the electrical activity of at least one brain region via EEG, i.e. measuring voltage fluctuations resulting from ionic current flows within the neurons of the brain region.

In another embodiment, the activity of at least one brain region is measured by measuring the blood flow in the brain region via functional magnetic resonance imaging (fMRI).

In a further embodiment, the activity of at least one brain region is measured by measuring the blood oxygenation in the brain region via functional near infrared spectroscopy (fNIRS).

It should be understood that any adequate method adapted to measure the activity of at least one brain region may be used. For example, magnetoencephalography, positron emission tomography, single-photon emission computed tomography, ultrasound methods, or the like may be used. In another example, measuring the temperature of the brain region may also be used to determine the activity thereof.

At step 18, the stimulus clarity perceived by the subject is assessed. The difference of perceived clarity is determined from the difference between the cerebral activity measured during the application of the first stimulus and the cerebral activity measured during the application of the second stimulus. It should be understood that the cerebral activity is caused by the integration of the sensory information, the decision making task related to the identification of the characteristic, and other activity sources such as memory, emotions, thoughts, etc. However, since the cerebral activity related to the decision making task and the other sources of activity is substantially constant during application of each stimulus, the difference of cerebral activity may be substantially related to the integration of the sensory information, and therefore to the subject's perception of the stimulus clarity. Therefore, the difference of cerebral activity between the first and second stimuli having different degrees of clarity indicates how the difference of clarity between the stimuli is perceived by the subject. The greater the difference of frontal cortex activity between the first and second stimuli is, the greater the subject perception of the stimuli clarity difference is.

In one embodiment, the stimuli are each applied a respective predetermined number of times to the subject. It should be understood that the time period during which the stimuli are applied to the subject may vary from one stimulus to another. Similarly, the time period between two successive stimuli may also vary. For example, a same first stimulus having a first degree of clarity may be applied a first predetermined number of times and a same second stimulus having a second and different degree of clarity may be applied to the subject a second predetermined number of times. The first and second predetermined number of times may be identical or different. In another embodiment, a first predetermined number of different first stimuli each having the same first degree of clarity are applied to the subject, and a second predetermined number of different second stimuli each having the same second degree of clarity are applied to the subject. The first and second predetermined numbers of stimuli may be identical or different.

In one embodiment, more than two different stimuli may be applied to the subject, so that more than two different stimulus degrees of clarity are presented to the subject. For example, a first reference stimulus having a first degree of clarity may be applied to the subject a first predetermined number of times, a second reference stimulus having a second degree of clarity greater than the first degree of clarity may be applied to the subject a second predetermined number of times, a third reference stimulus having a third degree of clarity greater than the second degree of clarity may be applied to the subject a third predetermined number of times, a fourth reference stimulus having a fourth degree of clarity being less than the first degree of clarity may be applied to the subject a fourth predetermined number of times, a fifth reference stimulus having a fifth degree of clarity being less than the fourth degree of clarity may be applied to the subject a fifth predetermined number of times, etc.

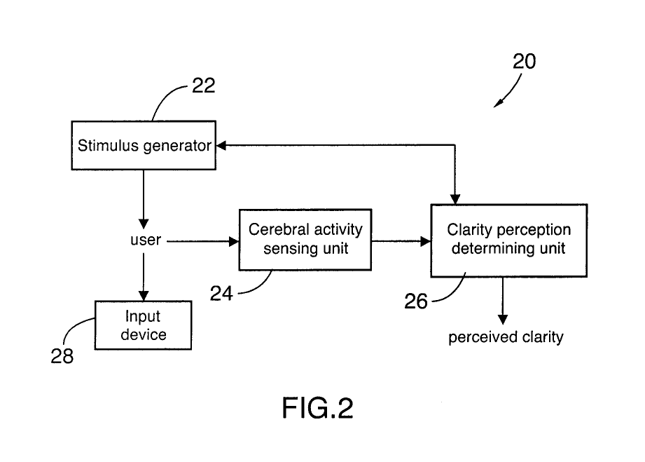

FIG. 2 illustrates one embodiment of a system 20 for assessing the clarity perception of a subject when sensory stimuli are applied to the subject. The system comprises a stimulus generator 22 for generating and applying stimuli to the subject, a cerebral activity sensing unit 24 for measuring the cerebral activity of at least one brain region of the subject, and a clarity perception determining unit 26 for determining the sensory stimulus clarity perceived by the subject.

The stimulus generator 22 is adapted to generate and apply to the subject at least two stimuli having different degrees of clarity. The cerebral activity sensing unit 24 is adapted to measure the activity of at least one brain region of the subject. The clarity perception determining unit 26 comprises at least a processing unit coupled to a storing unit and the processing unit is configured for determining the stimulus clarity perceived by the subject by comparing the cerebral activity measured during the application of the first stimulus to the cerebral activity measured during the application of the second stimulus, as described above.

In one embodiment, the stimulus generator 22 is connected to the clarity perception determining unit 26 to allow the clarity perception determining unit 26 to determine which parts of the measurement received from the cerebral activity sensing unit 24 correspond to the first stimulus and the second stimulus. For example, the stimulus generator 22 may send signal indicative that a stimulus is applied to the subject during the application of each stimulus. In another example, the stimulus generator 22 may send a timing signal to the clarity perception determining unit 26 at the start and the end of the application of each stimulus. In still another example, the time period of application of each stimulus may be stored on the clarity perception determining unit 26 and the stimulus generator 22 may be adapted to send a signal to the clarity perception determining unit 26 at the beginning of the application of each stimulus.

In another embodiment, the clarity perception determining unit 26 is further adapted to control the stimulus generator 22 and/or the cerebral activity sensing unit 24. In this case, the clarity perception determining unit 26 concurrently triggers to the application of the stimuli and the measurement of the frontal cortex activity.

In one embodiment, the cerebral activity sensing unit 24 is an EEG device comprising at least one electrode and adapted to measure the electrical activity of at least one brain region via EEG, i.e. to measure voltage fluctuations resulting from ionic current flows within the neurons of the frontal cortex. In one embodiment, the electrodes are each positioned at a respective position adequate for sensing the desired sensory cortex of the subject in order to measure the electrical activity of at least one region of the sensory cortex of the subject. In another embodiment, the electrodes are each positioned at a respective position adequate on the forehead of the subject for sensing the frontal cortex of the subject in order to measure the electrical activity of at least one region of the frontal cortex of the subject.

In another embodiment, the cerebral activity sensing unit 24 is an fNIRS device which comprises at least one optode and is adapted to measure the blood oxygenation of at least one region of the brain of the subject. In one embodiment, the optodes are each positioned at a respective position adequate for sensing the desired sensory cortex of the subject in order to measure the blood oxygenation of at least one region of the sensory cortex of the subject. In another embodiment, the optodes are each positioned at a respective position adequate on the forehead of the subject for sensing the frontal cortex of the subject in order to measure the blood oxygenation of at least one region of the frontal cortex of the subject.

In a further embodiment, the cerebral activity sensor 24 is a functional magnetic resonance imaging (fMRI) device adapted to measure the blood flow in the frontal cortex of the subject.

Any adequate system/method for positioning and holding the probes, e.g. the electrodes, the optodes, or the like, at an adequate position on the head of the subject may be used. For example, in the case of an EEG device, the electrodes may be removably secured directly on the forehead of the subject at adequate locations thereon.

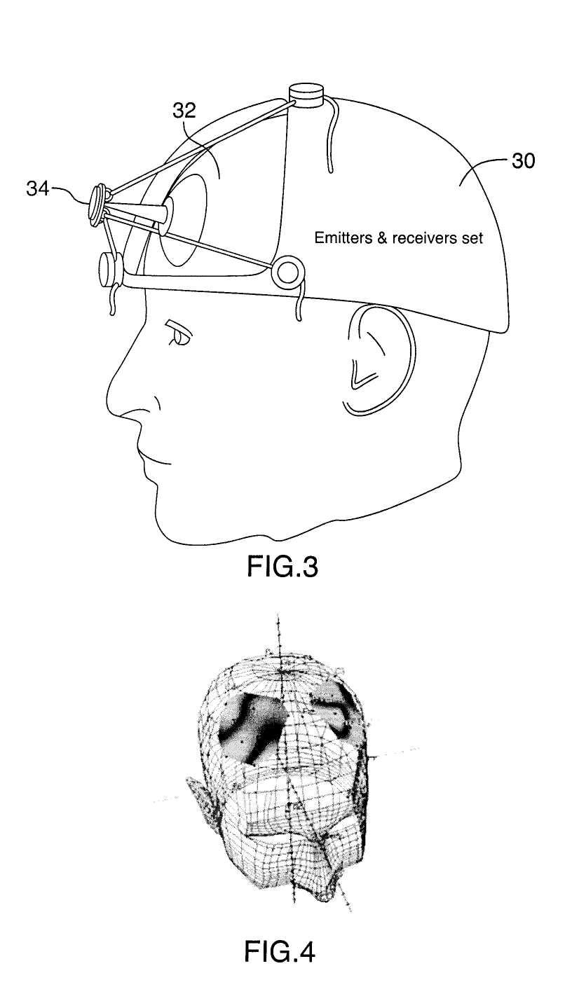

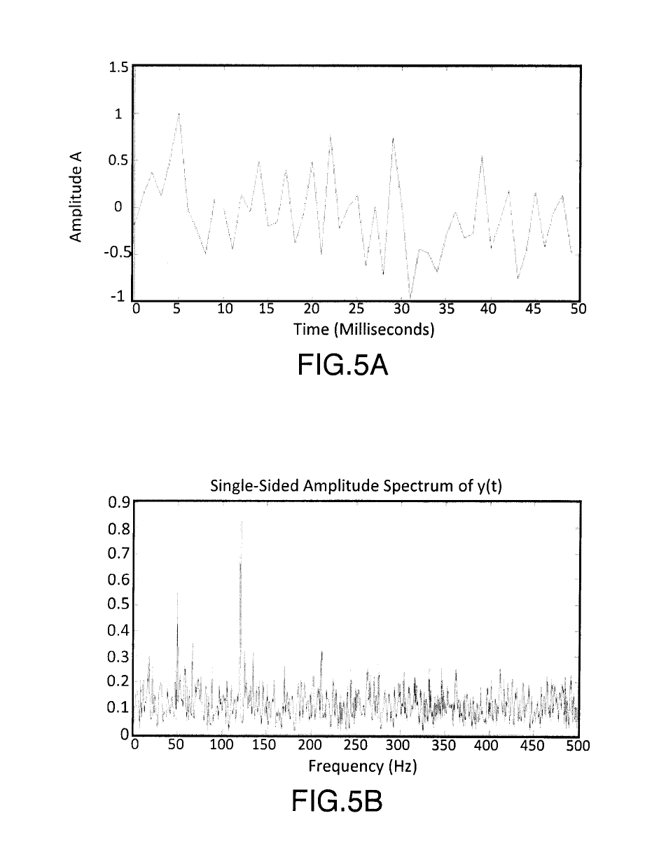

FIG. 3 illustrates a helmet 30 provided with two openings 32 each facing a different region of the forehead of the subject. The helmet 30 further comprises two optodes 34 each facing a respective opening 32. FIG. 4 illustrates the two frontal regions of which the activity is measured by the optodes 34.

In another embodiment, the probes may be secured to a frame on which the subject abuts his forehead.

In one embodiment, the system 20 further comprises a user input device 28 for allowing the subject to enter the characteristic of the stimuli. For example, the user input device 28 may be a touch-screen unit for displaying possible choices for the characteristic of the stimuli. In another example, the user input device may be a keyboard, a mouse, or any adequate input device comprising keys or buttons to be depressed by the subject for entering the characteristic of the stimuli. It should be understood that the user input device 28 may not be connected to the clarity perception determining module 26 since the ability of the subject to successfully identify the characteristic of the stimuli does not influence the determination of his clarity perception.

It should be understood that the above method and system 10 and 20 may be used for assessing the clarity perception of a subject for any one of a visual stimulus, an auditory stimulus, a tactile stimulus, an olfactory stimulus, or a gustatory stimulus.

In one embodiment, the clarity of the stimuli is changed by varying the frequency spectrum of a reference natural stimulus. Natural stimuli can be characterized by a range of frequencies (or components) of differing amplitudes. When the amplitudes are modified (or other filtering process), then the degree of clarity of the stimulus is changed. This has been established and holds for all sensory modalities.

In an embodiment in which visual clarity of a subject is to be assessed, the visual stimuli comprise natural images, which contain various frequencies with different amplitudes. By modifying the frequency amplitude spectrum (e.g. filtering), one can change the clarity of an image, so that the image appears blurry or overly-detailed (sharpened). The image perceived as clearest, is generally the original natural image (or one very close to it). A series of natural images with varying levels of clarity are displayed to the subject on a display unit. The subject is asked to specify whether the image belongs to one of two categories of equivalent difficulty: for example, whether the images contain a cat or a dog, a triangle or a circle, etc. In one embodiment, the frontal cortex activity is then measured and the collected data are analyzed to determine differences in perceived visual clarity. In another embodiment, the activity of the visual cortex which comprises occipital brain regions is measured and the collected data are analyzed to determine differences in perceived visual clarity between the different visual stimuli.

It should be understood that any adequate visual stimuli of which the clarity may be varied may be used. For example, lights of varying intensity or color may be used.

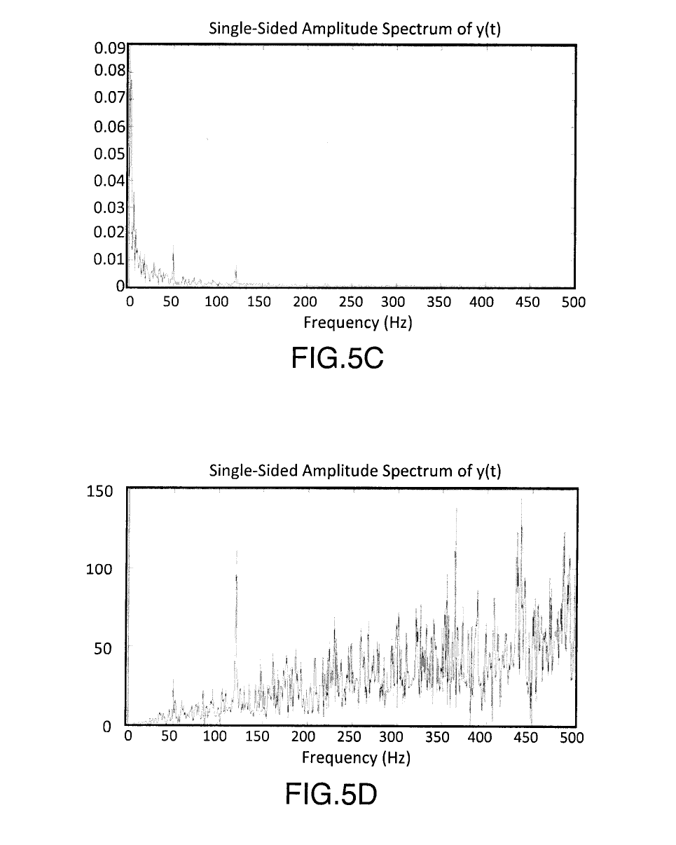

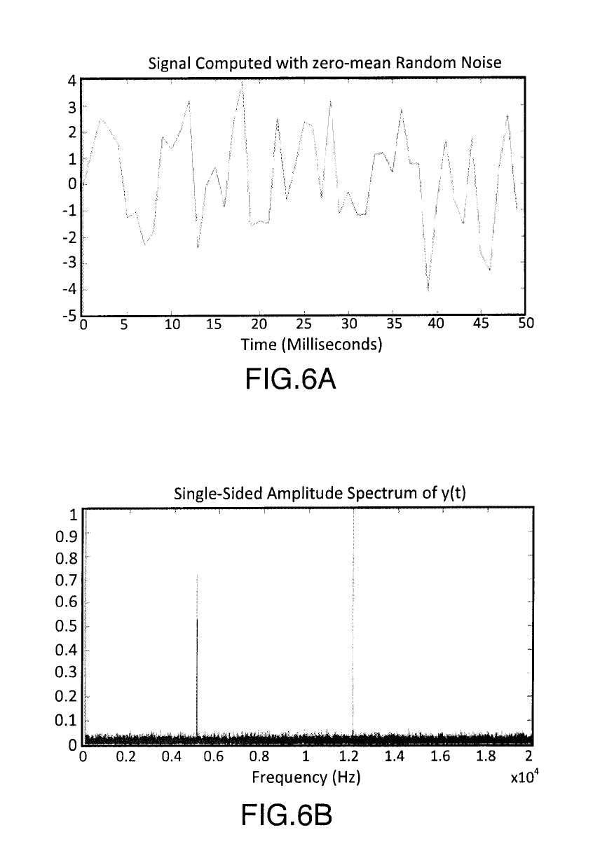

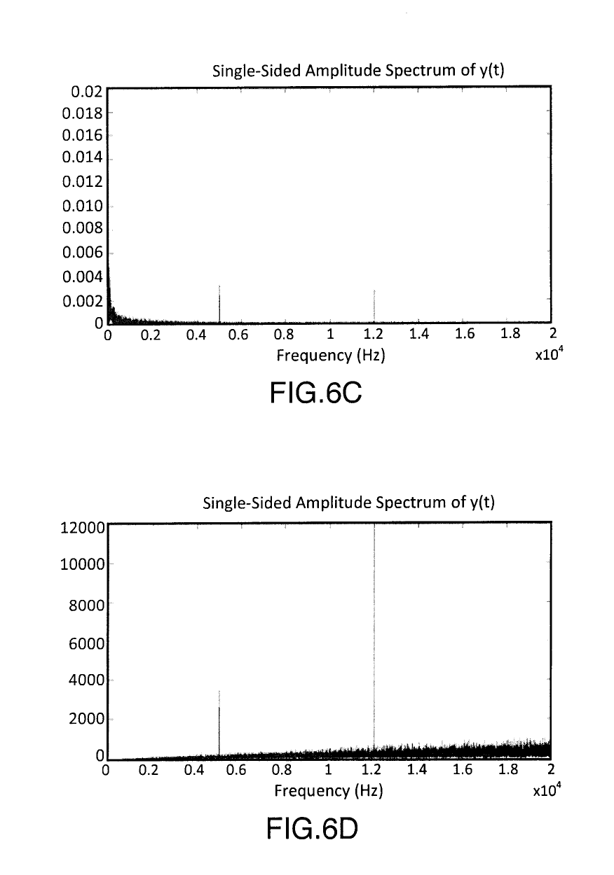

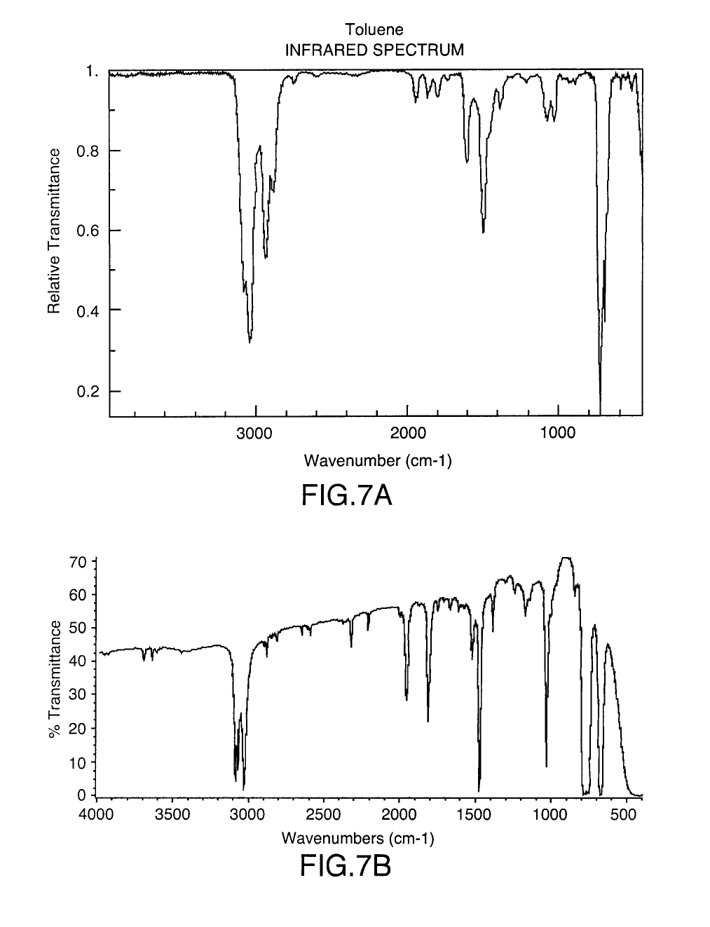

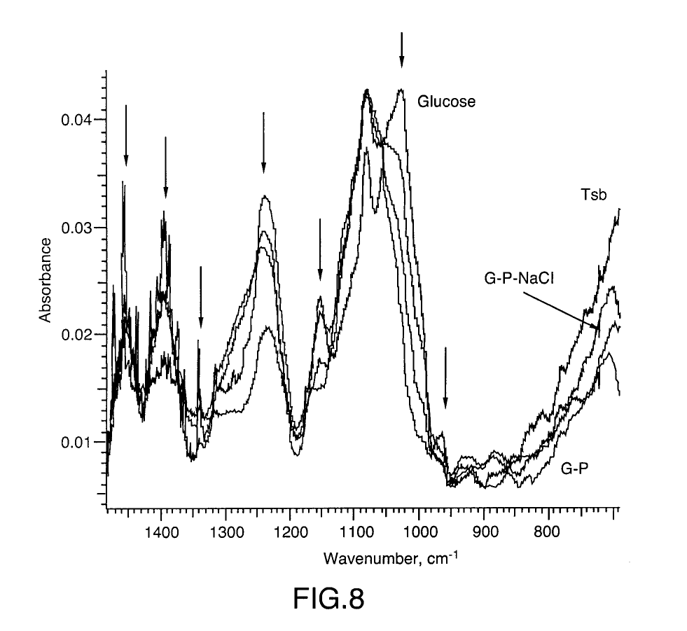

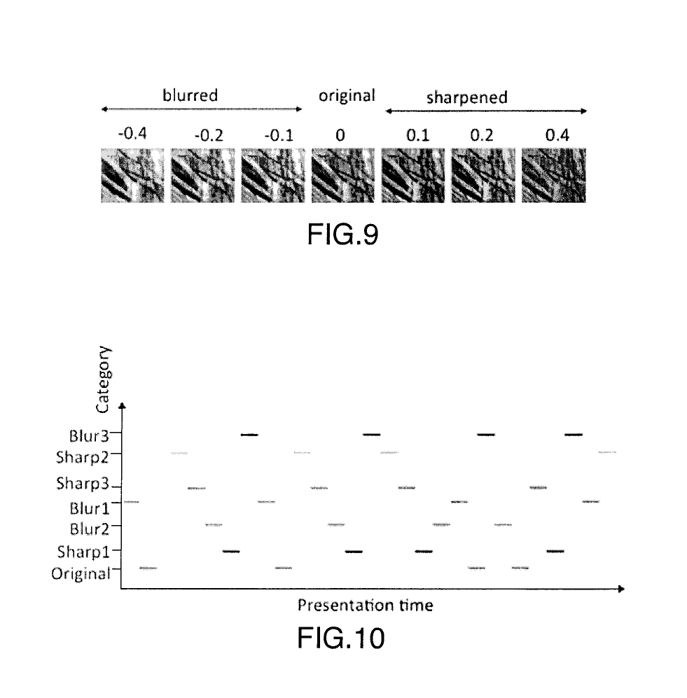

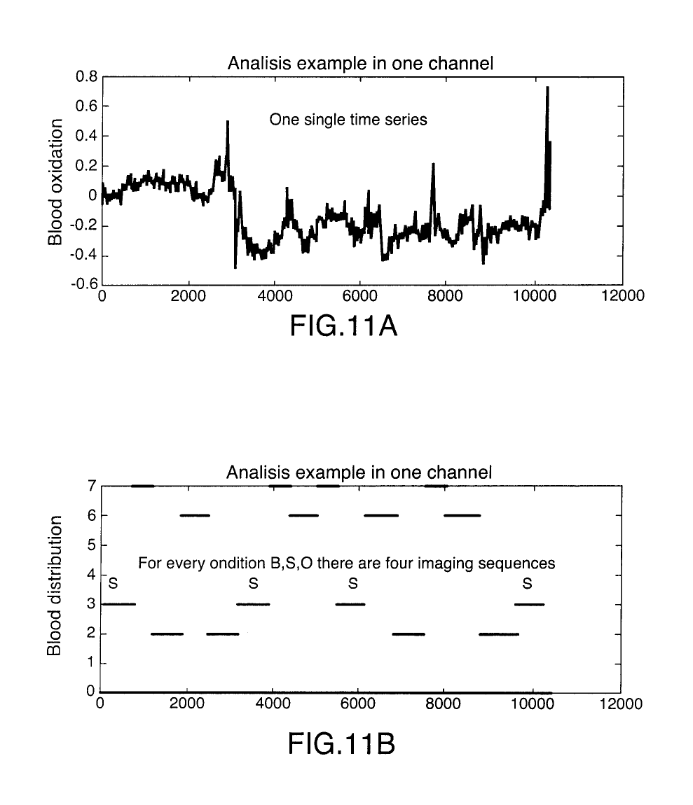

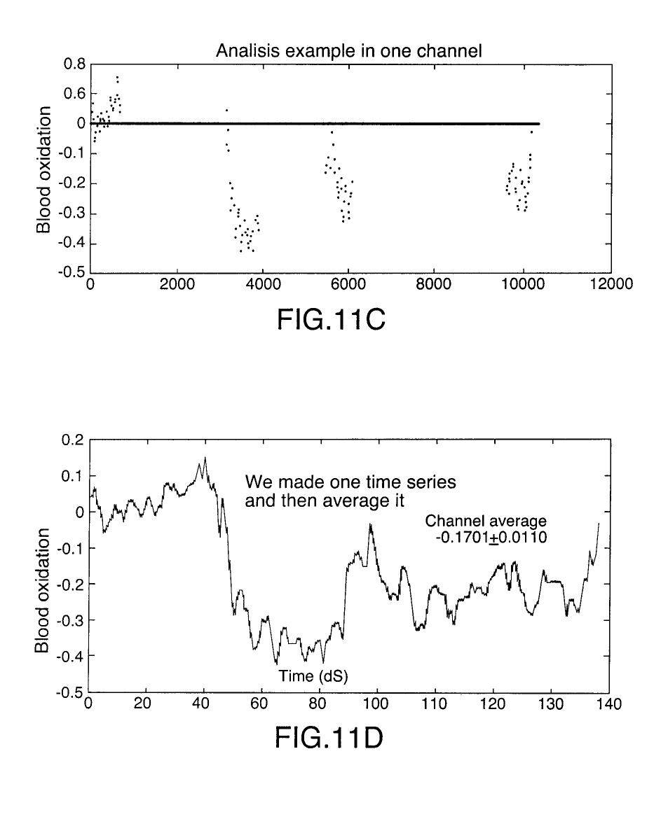

In an embodiment in which auditory clarity of a subject is to be assessed, auditory stimuli comprising sounds of varying clarity are applied to the subject. Similarly to natural images that contain frequencies of differing amplitudes, sounds are also composed of frequencies of differing amplitudes. As in the visual case, a sound's spectrum can be modified (e.g. filtered) to have varying degrees of clarity. FIGS. 5a-5d illustrate different exemplary auditory stimuli. FIG. 5a illustrates a time series with amplitude (y-axis) plotted as a function of frequency (x-axis) for an original auditory stimulus. FIG. 6b illustrates a power spectrum distribution of the original stimulus. FIG. 5c illustrates an exemplary power spectrum distribution for a blurred auditory stimulus, in which power is plotted as a function of frequency in Hz. FIG. 5d illustrates an exemplary power spectrum power distribution for a sharpened auditory stimulus, in which power is plotted as a function of frequency in Hz. In this example, the signal contains a 5 KHz sinusoid and 12 KHz sinusoid corrupted with some zero-mean random noise.

In the auditory modality, a series of sounds with varying degrees of clarity are presented to the subject via speakers, headphones, in-ear monitor (IEM) earpieces, etc. The subject is requested to identify a characteristic of the sounds. For example, the subject may be asked to specify which of two categories the sounds belongs to (for example a cat meowing or a dog barking). In one embodiment, the frontal cortex activity is then measured and the collected data are analyzed to determine differences in perceived auditory clarity. In another embodiment, the activity of the auditory cortex, which comprises temporal brain regions is measured and the collected data are analyzed to determine differences in perceived auditory clarity between the different auditory stimuli.

In an embodiment in which tactile clarity of a subject is to be assessed, tactile stimuli such as any surface or object can also be described by a spectrum of frequencies, which can be modified to have varying degrees of clarity. FIGS. 6a-6d illustrate different exemplary tactile stimuli. FIG. 6a illustrates a time series with amplitude (y-axis) plotted as a function of frequency for an original tactile stimulus. FIG. 6b illustrates a power spectrum distribution of the original stimulus. FIG. 6c illustrates an exemplary power spectrum distribution for a blurred tactile stimulus, in which power is plotted as a function of frequency in H. FIG. 6d illustrates an exemplary power spectrum power distribution for a sharpened tactile stimulus, in which power is plotted as a function of frequency in Hz.

In the tactile modality, a series of patterns with varying degrees of clarity is presented to the subject. The subject is requested to identify a characteristic for each stimulus. For example, the subject may be asked to specify which of two fingers (analogous to categories) is touched by the stimulus. In another example, the subject may be asked to determine and specify whether the pattern that touches his finger is a cross or a circle. In one embodiment, the frontal cortex activity is then measured and the collected data are analyzed to determine differences in perceived tactile clarity. In another embodiment, the activity of the tactile cortex which comprises frontal and parietal brain regions is measured and the collected data are analyzed to determine differences in perceived tactile clarity between the different tactile stimuli.

For example, the stimulus generator may consist of objects with small embossed shapes (e.g. a cross and a circle). The shape's edges are either neat or degraded (corresponding to blurred) and the subject is requested to characterize which of the two shapes they touched. In another example, the stimulus generator may comprise a single pin-point object that may be applied with varying degrees of pressure (weak pressure corresponds to blurred, moderate to original, and strong to sharpened) on one of two fingers, and the subject is requested to specify which of two fingers was touched.

It should be understood that any adequate tactile stimuli of which the clarity may be carried may be used. For example, the temperature of a heating/cooling object may be varied to apply stimuli of varying temperatures.

In an embodiment in which olfactory clarity of a subject is to be assessed, olfactory stimuli such as a scent or an odor can be described by a spectrum of components with varying amplitudes (or intensities). This spectrum can be modified to have varying degrees of clarity for a particular scent.

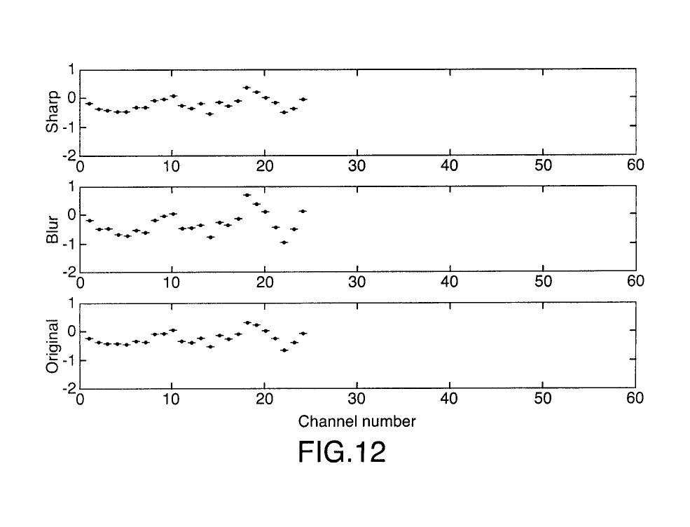

FIGS. 7a and 7b illustrates examples of Fourier transform of infrared spectra showing peaks for toluene (FIG. 7a) and benzene (FIG. 7b). In one example, the original solution would have equal concentrations of toluene and benzene. An example of blurring occurs when more toluene than benzene is added, and of sharpening when benzene is higher than toluene. The toluene and benzene are only examples of how to implement the experiment, and would not be used in an actual experiment with human participants.

In the olfactory modality, a series of scents with varying degrees of clarity is presented to the subject. The subject is requested to identify a characteristic for each olfactory stimulus. For example, the scent can be directed towards one nostril or the other, and the subject is asked to specify whether he smells the scent in the left or right nostril. In one embodiment, the frontal cortex activity is then measured and the collected data are analyzed to determine differences in perceived olfactory clarity. In another embodiment, the activity of the olfactory cortex which comprises temporal brain regions is measured and the collected data are analyzed to determine differences in perceived olfactory clarity between the different olfactory stimuli.

In one example, an apparatus applies an odor to one nostril at the moment of inhalation, so that it is “absorbed” by the nose. Stimulus odor clarity is accomplished by titrating between two odors (including a zero point where both are equally strong, corresponding to the point of subjective equality). The subject is requested to specify which of two nostrils was presented with the odor. Another example involves diluting a single odor, but a strong neutralizing odor (such as coffee for example) is presented between each stimulus presentation, to prevent adaptation to the single odor. In both scenarios, instead of presenting odors to one of two nostrils, the odors can arise from one of two locations and the subject is requested to specify the location where the scent came from.

In an embodiment in which taste clarity of a subject is to be assessed, gustatory stimuli such as solutions composed of numerous components with varying amounts of each component can be applied to the subject. By maintaining the total amount of components, but by changing the proportion of each component, stimulus clarity can be varied.

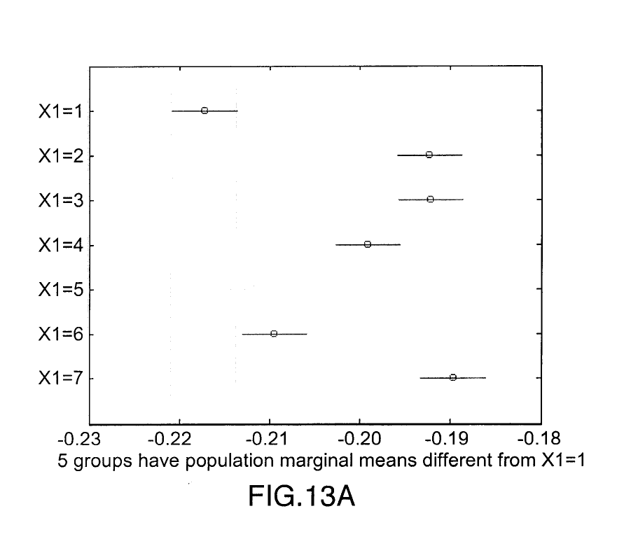

FIG. 8 illustrates one example of a Fourier transform of infrared spectra showing the peaks of glucose (sugar) and sodium chloride (salt) for a gustatory stimulus. In this case, the original solution would be a solution with equal amounts of salt and sugar. An example of blurring occurs when more sugar than salt is added, and of sharpening when the salt concentration is higher than the sugar concentration.

In the taste modality, different solutions with different levels of taste clarity are presented to the subject who iteratively tastes them. The subject is requested to identify a characteristic for each gustatory stimulus. For example, the solutions can be placed on the left or right of the subject's tongue, and the subjects may be asked to specify on which side of his tongue the solution has been placed. In one embodiment, the frontal cortex activity is then measured and the collected data are analyzed to determine differences in perceived gustatory clarity. In another embodiment, the activity of the gustatory cortex which comprises temporal and parietal brain regions is measured and the collected data are analyzed to determine differences in perceived taste clarity between the different gustatory stimuli.

In one embodiment, the stimulus generator comprises a surface with two openings, which is placed on the tongue of the subject and a switch is used to drop a solution on one of the two openings. For example, the solution's taste clarity is modified by titrating between two tastes (e.g. salt and sugar). The participant is requested to specify which of the two openings received the solution. In another example, a single taste is used (e.g. salt only) and the solution is diluted to have varying degrees of clarity.

The following describes an application of the above method 10 and system 20 for determining which one of two corrective lenses is better adapted to a subject. The subject is asked to iteratively look at a display unit through at least two different corrective lenses. Images of varying image clarity are displayed on the display unit while the subject looks at the display unit through each corrective lens. For each corrective lens, the method 10 is applied in order to determine a respective difference of visual clarity perceived by the subject between the images of varying clarity. The corrective lens that is the most adapted to the subject is identified as being the one that has the greatest difference of visual clarity perceived by the subject.

In this case, the stimulus generator 22 comprises a display unit for displaying the images, a storing unit on which the images of varying degrees of clarity are stored, and a processing unit for retrieving the images from the storing unit and transmitting them to the display unit.

It should be understood that any adequate method for analyzing the cerebral activity measurement data in order to determine the difference of perceived clarity between two stimuli may be used. In the following exemplary methods are described for a particular case when the method 10 is used for assessing perceived visual clarity.

Let be a physical system that is determined by a function state L that depends on independent extensive parameters Xi (i=1, 2, . . . n) corresponding to the cerebral activity data measured from at least one brain region. The parameters Xi may depend on the time tj (j=1, 2 . . . o) with Dt=t(j+1)−tj corresponding to the inverse of the sampling frequency. The state of the system is measured by using an external interface. The interface may be classified as passive or active. For a passive interface, it is not necessary to introduce into the system any sort of energy, e.g. light, voltage, current. The interface includes l (l=1, 2 . . . p) probe-detectors (active interface) or N detectors (passive interface) that herein we will call them probes. The interface is designed to operate at a certain sampling rate that is established according with the system dynamics.

By considering the interaction of the system with an external stimulus condition Ck (k=1, 2 . . . q), it is possible to determine how the parameters Xijkl change accordingly. For example, a subject can be asked to look a series of different visual stimuli each corresponding to a condition Ck. This in principle triggers a series of events within the system that the probes measure. At the end, a matrix formed by the parameters Xijkl is obtained.

In one embodiment, a statistical analysis is performed on the measurement data, e.g. the blood oxygenation data, the electrical activity data, to determine the difference of perceived clarity between the stimuli of different clarity.

At a first step and in order to analyze if the state of the system has changed for every condition, each parameter Xijkl is averaged for all j and for each i-category; the pairs Xkl are compared together. In one embodiment, the first step can be performed using a t-test with Bonferroni corrections. In another embodiment, the first step can be performed using an analysis of variance (ANOVA) method. At a second step, the change of the state can be determined from the differences between different k,l's. The comparisons also allow determining which channels l are the most sensitive to show differences among different conditions k.

In another embodiment, the energy or power distribution of the parameters Xijkl is determined to characterize the difference of perceived clarity between the stimuli of different clarity. For each parameter Xijkl, the energy or power distribution is determined by performing a harmonic analysis. Then, the statistical analysis described above is repeated. In this case, the L function depends on the energy associated with the system.

In a further embodiment, the entropy of the parameters Xijkl is determined to characterize the difference of perceived clarity between the stimuli of different clarity. In this case, the coefficients i,k,l are fixed for each parameter Xj and a configurational space is generated by shuffling the order of each j by following the below described rules, and then create a function L that depends on how many of the new configurations Xj′ remain similar. If the parameters Xj′ have substantially high irregular fluctuations, then the function state L is expected to be large if the harmonic content is large (when the noise is short). In this case, the L function depends on the entropy associated with the system.

Then different analysis methods may be used. In one embodiment, the statistical analysis described above is applied, but instead of taking the average over all j's, the parameter Xijkl is determined for each i-category. In another embodiment, a multiscale time analysis of the function L is performed. This is done by repeating the determination of the above described configurational space while using different time windows. For example, the method may start by setting Xj=3, and then L(Xj=3) is calculated. The coefficient j is then increased, and the steps are repeated for Xj=4, Xj=5 etc, till j=m. Then for each i-condition, we look for a time window with label j″, where a subset of channels l′ show different L(Xj″kl′) values for all the values of k.

It should be understood that any adequate state function for analyzing the cerebral activity measurement data in order to determine the difference of perceived clarity between two stimuli may be used. For example, Massieu functions or any other adequate thermodynamical potential may be used.

There is described below an example of the application of the three above described analysis methods.

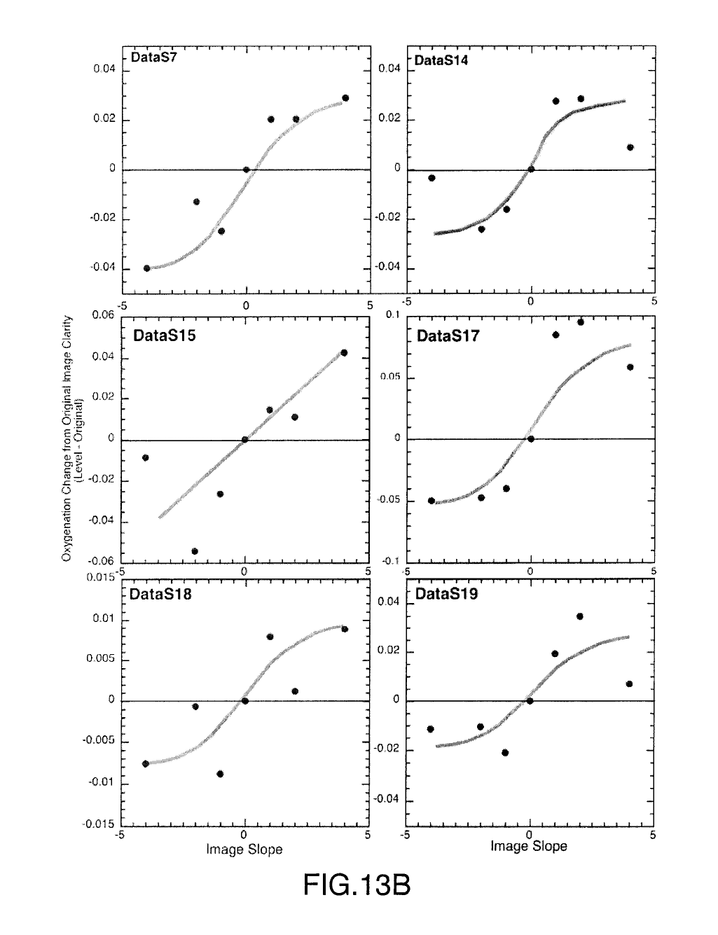

Seven different visual conditions/stimuli are presented to a subject. The stimuli comprise an original image, three blurred images having different levels of clarity, and three sharpened images having different levels of clarity, as illustrated in FIG. 9.

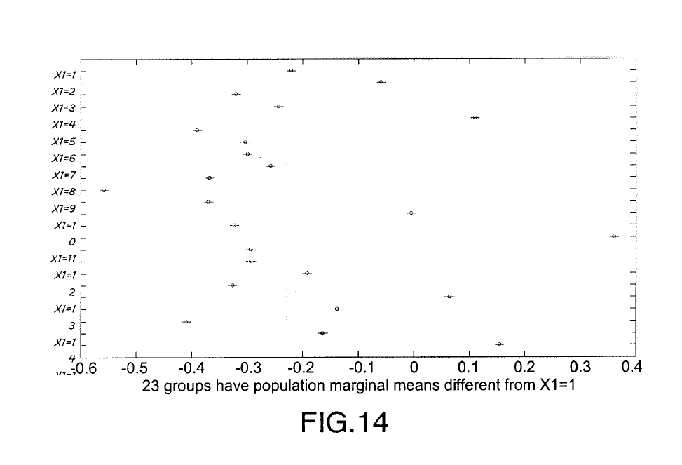

The visual conditions/stimuli are applied to the subject in a block series as illustrated in FIG. 10.

The oxygenation of blood of 24 regions of the frontal cortex is measure by means of NIRS, thereby providing 24 data channels. Every channel measures the seven conditions.

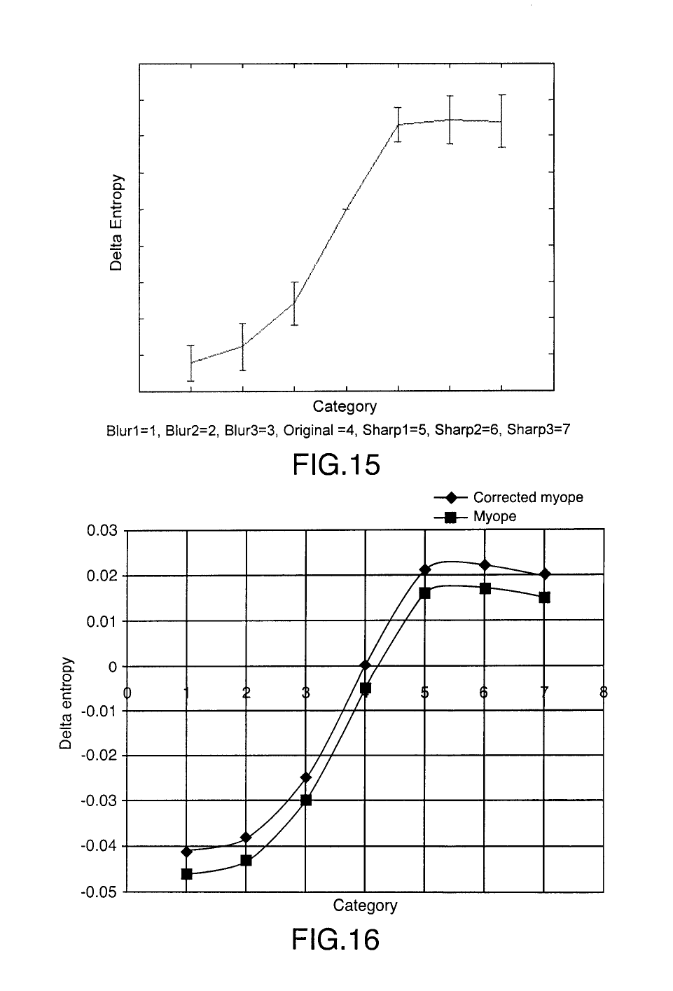

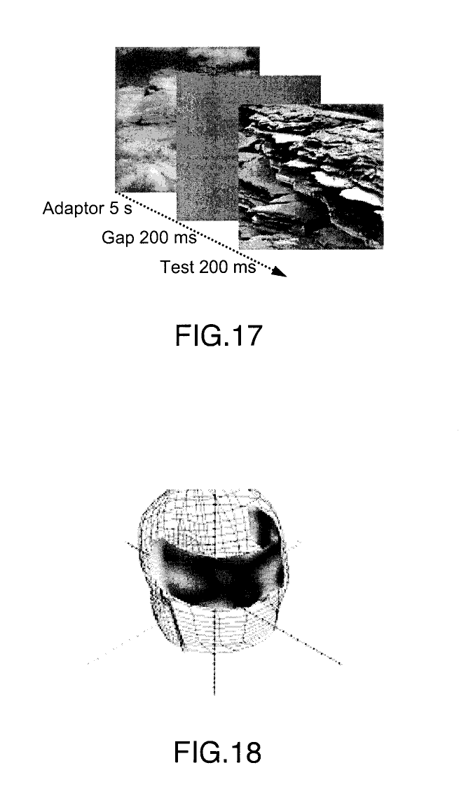

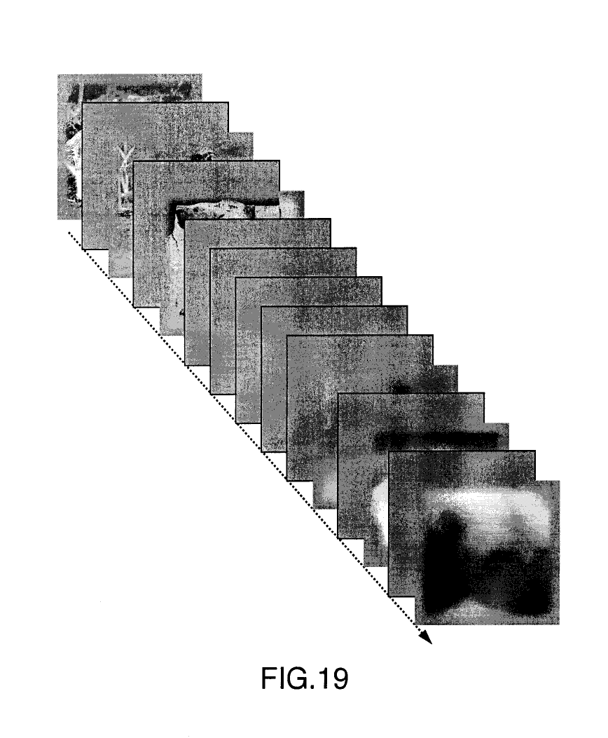

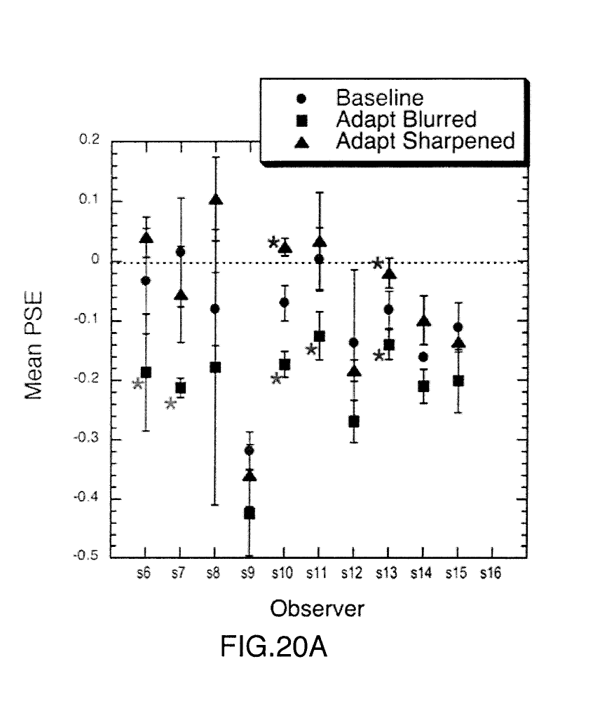

FIG. 11a illustrates the blood oxygenation in time corresponding to a given one of the 24 channels while FIG. 11b identifies the location in time of the sharpened condition/stimulus having a first sharpened level of clarity. FIG. 11c illustrates the blood oxygenation in time corresponding to the sharpened condition/stimulus having the first sharpened level of clarity. FIG. 11c comprises four sets of data separated in time. FIG. 11d illustrates the four sets of data of FIG. 11c regrouped together to form a single time sequence, which represents the blood oxygenation in time for only the sharpened condition having the first sharpened level of clarity.

In this case, the function Xi represents the blood oxygenation (i=1) that depends on time. The time series for each visual condition runs from j=1, 2 . . . (10600/4), Dt=1/10 (10 Hz is the sampling frequency).

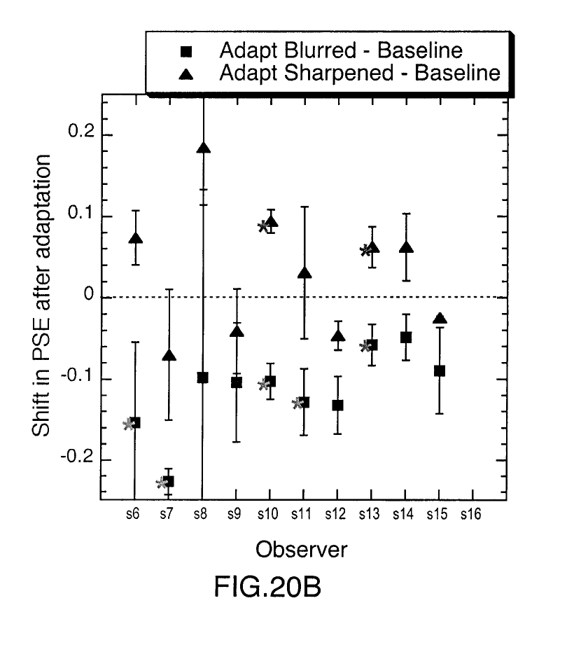

In one embodiment, the above described statistical analysis is applied to the blood oxygenation data. In this case, for each one of the 24 channels and for each condition, the blood oxygenation, such as the one illustrated in FIG. 11d, is averaged in time, thereby obtaining 24 average blood oxygenation values for each condition. FIG. 12 illustrates the average blood oxygenation value as a function of the channels for three exemplary conditions, i.e. original, sharpened no. 1 and blurred no. 1.

Then, a t-test may be applied to compare them and discriminate if the conditions are different among them.

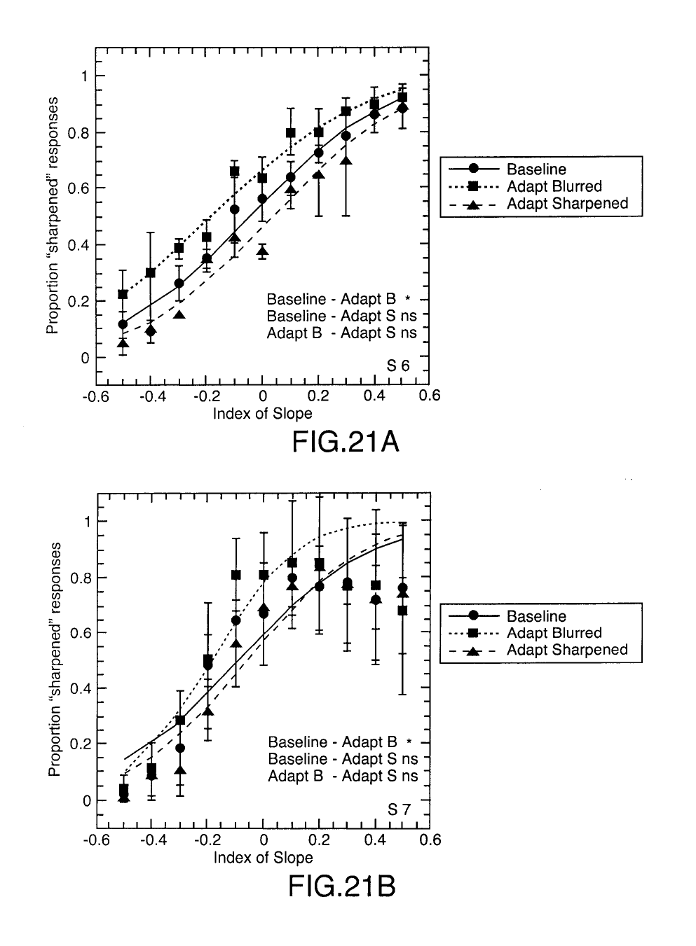

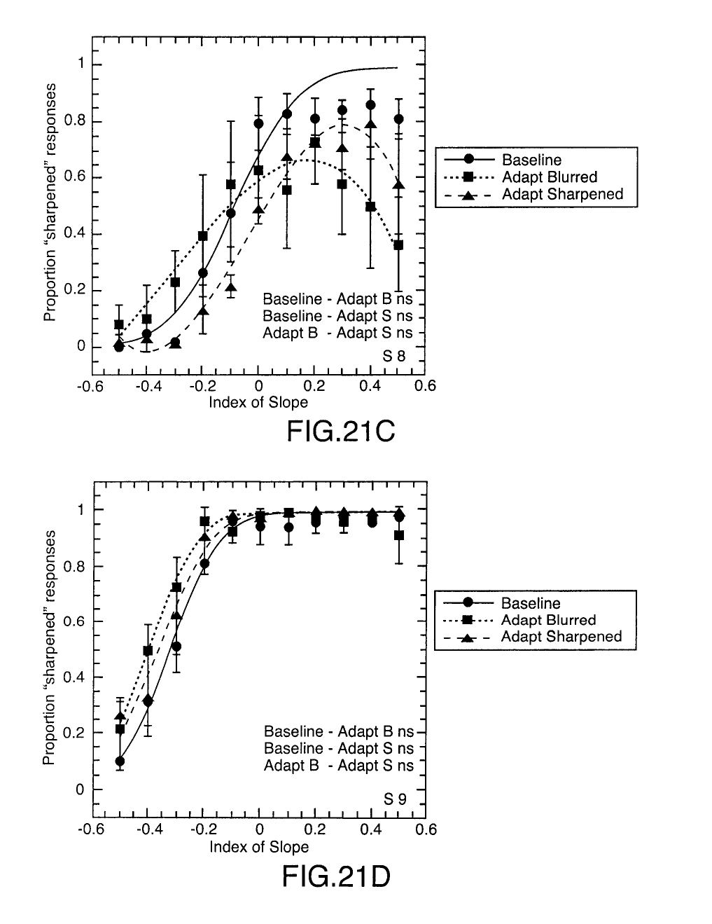

An ANOVA analysis may also be performed to determine differences between channels by comparing the means of all conditions. In this case, the blood oxygenation averaged over time is further averaged over the channels so that a single average blood oxygenation is obtained for each condition, as illustrated in FIG. 13a. FIG. 13b illustrates the oxygenation change as a function of a variation of image clarity level. Those that are closest to the original represent the finest (most subtle) difference from the original. Curves with the largest difference in oxygenation (y-axis) for these near-original levels (x-axis) represent the best clarity conditions for an individual.

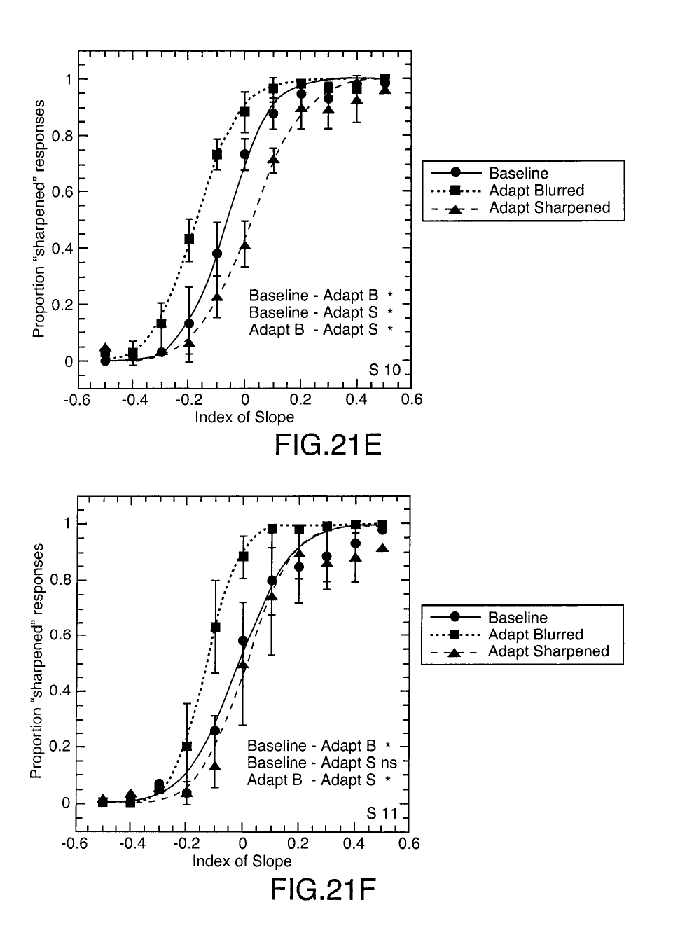

The ANOVA method may also be used for identifying differences between channels by comparing the means of all conditions as illustrated in FIG. 14. Each point represents the mean of all conditions at every channel. In this way, it is possible to determine which channels present different activity and which ones present the same activity. Therefore we can filter out the number of channels may be filtered out, and it can also be determined which cortical region is activated most with the task.

In another embodiment, the energy or power distribution of the blood oxygenation data is determined. Power spectral density (PSD) describes how the power of a signal or time series is distributed with frequency. In the present case, power can be the actual physical power, or more often, for convenience with abstract signals, can be defined as the squared value of the signal. This instantaneous power is then given by:

P(t)=f(t)²

for a signal f(t). The mean (or expected value) of P(t) is the average power, which is related to the power spectral density evaluated at zero frequency,

<P(t)>=PSD(ω=0).

More generally, a normalized Fourier transform for ω=2πn/T is defined:

$${F}_{T} ( ω ) = \frac{1}{\sqrt{T}} {\int }_{0}^{∞} f ( t ) exp ( - i ω t ) d t$$

and the power spectral density is defined as:

PSD(ω)=F_{T}(ω)F*_{T}(ω)

where * means conjugate.

In the case of a finite time-series such as Xijkl, we obtain:

$$PSD ( ω ) = \frac{{Dt}^{2}}{T} \vert {\sum }_{j = 1}^{o} {X}_{ijkl} {e}^{- i ω \sqrt{- 1}} \vert ,$$

with T=oDt.

In one embodiment, the statistical analysis presented above may also be applied to the power spectral densities.

In a further embodiment, the entropy is used for characterizing the perceived visual clarity.

The algorithm to calculate the entropy is:

Step 1. Form a time series of data u(1), u(2), . . . , u(N). These are N raw data values from measurements equally spaced in time.

Step 2. Fix m, an integer, and r, a positive real number. The value of m represents the length of compared runs of data, and r specifies a filtering level.

Step 3. Form a sequence of vectors x(1), x(2), . . . , x(N−m+1) in R^{m}, real m-dimensional space, defined by x(i)−[u(i), u(i+m−1)].

Step 4. Use the sequence x(1), x(2), . . . , x(N−m+1) to construct, for each I, 1≦i≦N−m+1,

Cᵢ^{m}(r)=(number of x(j) such that d[x(i),x(j)]≦r)/(N−m+1).

We must define d[x(i),x(j)] for vectors x(i) and x(j). We follow the Takens modification of the formula as given in ref. 5 by defining

$$d [ x , {x}^{*} ] = {max}_{a} \vert u ( a ) - {u}^{*} ( a ) \vert ,$$

where u(a) are the m scalar components of x. d represents the distance between vectors x(i) and x(j), given by the maximum components.

Step 5. Next define

$${Φ}^{m} ( r ) = {( N - m + 1 )}^{- 1} {\sum }_{i = 1}^{N - m + 1} ln {C}_{i}^{m} ( r ) ,$$

Where ln is the natural logarithm.

Through step 5, the K-S entropy and ApEn algorithms are identical. The next step distinguishes between K-S entropy and ApEn.

Step 6 (K-S)

$$K \text{-} S entropy = {lim}_{r \to 0} {lim}_{m \to ∞} {lim}_{N \to ∞} [ {Φ}^{m} ( r ) - {Φ}^{m + 1} ( r ) ]$$

This is an abstract formula, rather than an algorithm, since we have not indicated an explicit procedure to take the limits and have not guaranteed the existence of a limit. Furthermore, close examination of this formula will convince the reader that the time to compute the expression within the brackets of this equation grows exponentially with m, compounding the difficulty of approximating the limit.

We can explicitly compute ApEn.

Step 6 (ApEn). We define approximate entropy by

ApEn=Φ^{m}(r)−Φ^{m+1}(r)

for m and r fixed as in step 2.

Typically, we choose m=2 or m=3; r depends greatly on the application.

In the present case, the U(i) series are the Xijkl, m=2 and r=0.15*std(Xijlk).

For the multiscale analysis we repeat the algorithm just describe above but instead of running from j=1, 2, . . . o., we use times subseries as j′=1,2,3. Then j″=1,2,3,4. Then j′″=1,2,3,4,5. The best time window is the one that shows significance difference between all the conditions k and for a subset of channels l or the whole set.

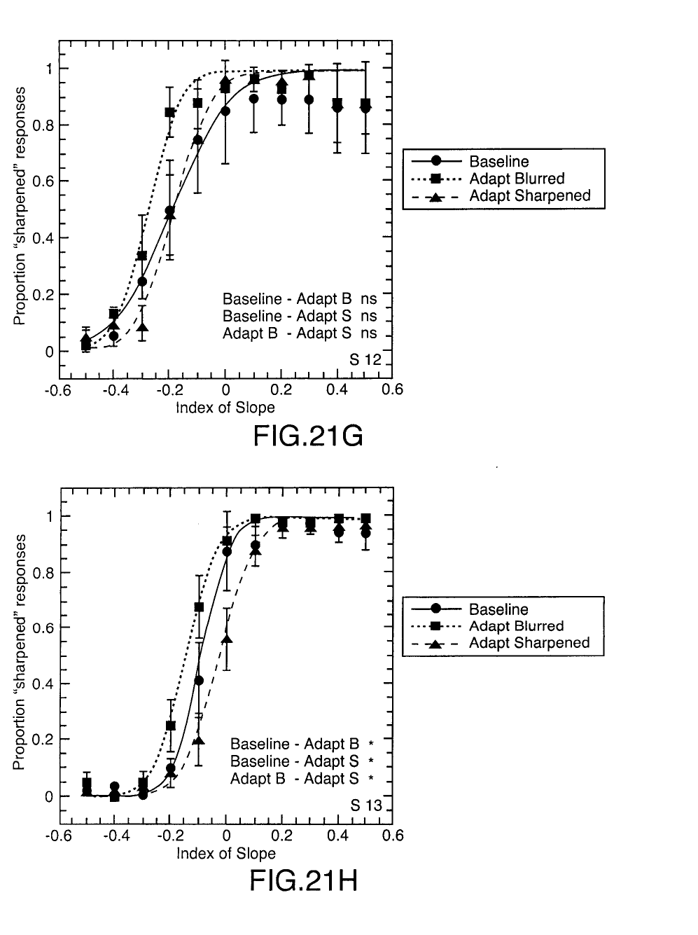

The entropy is determined according to the above-described method for each condition and channel. Taking the entropy for the original condition as a reference, we subtract it from the blur and sharp images entropy and retain only the values that fulfill the criteria entropy original>entropy blur and entropy original<entropy sharp. Then, we average out all these subtractions among all the channels and obtain the graphic illustrated in FIG. 15, where the vertical axe represents the differential entropy with the original entropy as a reference and the horizontal axis represents the seven visual conditions.

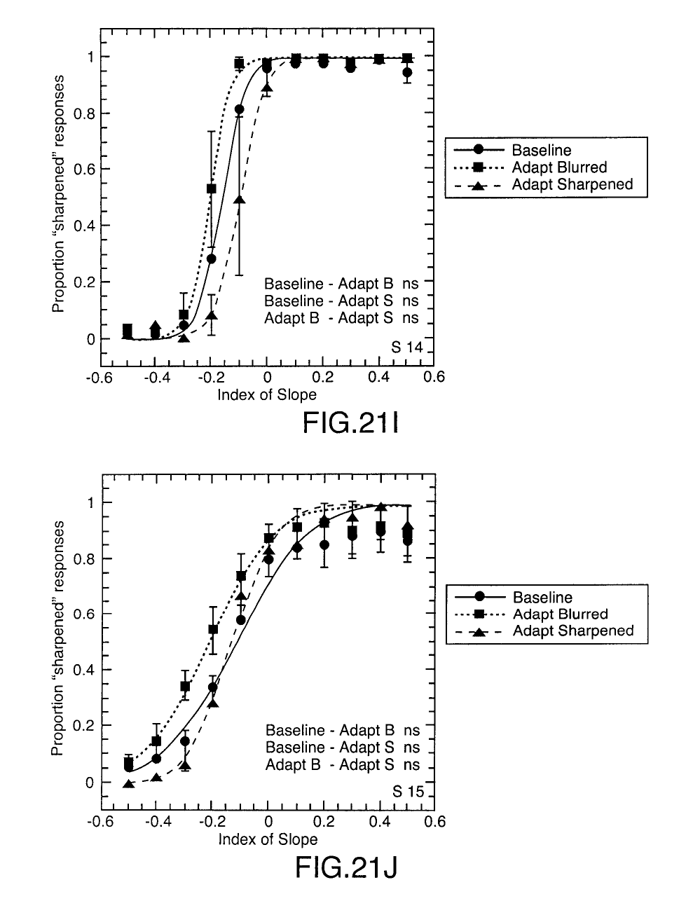

FIG. 16 illustrates the differential entropy for a myopic subject with and without corrective lenses. The curve illustrated in FIG. 16, which is shifted away from the center and towards the negative range (left), is typical of myopia.

The following presents experimental results for the characterization of visual clarity.

Adaptation is often observed by measuring human responses to a test image after prolonged exposure to an adapting image. After adapting to a blurred image, subsequent images appear sharper, and adapting to a sharpened image makes subsequent images seem blurred. For images that are only slightly blurred or sharpened, adaptation functions are fairly symmetrical and consistent across observers. However, when extending the blurred-sharpened range, these adaptation functions can exhibit asymmetry and significant individual variability. The variability in these adaptation functions could arise from differences in optical aberrations, in tolerance to blur, and even in personality. Furthermore, additional variability can arise from participant experience, where observers experienced in visual testing display more consistent results than their inexperienced counterparts, as in measures of vergence and accommodation for example.

With various factors influencing adaptability and because of its clinical importance, we sought to assess adaptation to blur on perceived image clarity in naïve observers with little or no experience in behavioral testing. Our approach was to replicate previous psychophysical work with an extended range of blurred-sharpened test images and to obtain an objective physiological measure of brain responses using fNIRS. We were interested in relevance to the general population and chose NIRS because of its broader applicability than techniques with better resolution but also greater constraints, such as fMRI or EEG (less portable and electrode placement is less comfortable-gels, hair washing).

### EXPERIMENT 1

Methods

Participants

Ten observers participated in this study and had normal or corrected-to-normal acuity. Participants consisted of 7 males and 3 females and were between 25 and 59 years of age.

In many studies of this type, participants are recruited from the department and are usually naïve to experimental goals, but they often consist of highly experienced psychophysical observers. We wanted to ensure a sample that better reflected the general population, and so, participants were recruited from a broader area (other university departments, general population).

Apparatus and Stimuli

Stimuli were generated on a Dell PC and presented on a calibrated CRT monitor (Sgi) using E-prime for the psychophysical runs and custom routines in C# for the NIRS experiments. Contrasts were linearized and the monitor was viewed binocularly from a distance of 114 cm (one pixel subtended 1.1 arc sec at the resolution of 1024×768) with a refresh rate of 75 Hz.

High resolution images of natural scenes were used. Color images were first converted to grayscale and then cropped to 500×500 pixels (subtending 9.2×9.2 degrees). Image blur or sharpening was manipulated through the following: an image can be defined by its Fourier amplitude spectrum (averaged across all orientations):

f∂f^{-s}

where f is the spatial frequency and s indicates the slope parameter, as the relationship is linear on a log-log plot. Images of natural scenes from our database had slopes ranging between −0.65 and −1.4. When image slope is modified from the original, images appear either blurred or sharpened. Images of natural scenes illustrated in FIG. 9 were processed to provide varying degrees of image clarity on an index scale ranging between −1 and 1, corresponding to blurred (−) and sharpened (+), respectively; 0 refers to the original image clarity. Root mean square (rms) contrast of the image was adjusted after slope changes so it could be maintained compared to the original image. Finally, image edges were softened by a Gaussian function, so that image contrast ramped smoothly down to the mean gray background.

Procedure

Behavioral: Psychophysics. Experiments were conducted using the method of constant stimuli and a single-interval forced-choice paradigm, in which observers were required to judge whether the image appeared blurred or sharpened. The point at which responses are divided equally between blurred and sharpened (50%) corresponds to the point of subjective equality (PSE).

Baseline. In the baseline condition, a test image was presented for 200 ms. Observers indicated whether the image appeared blurred or sharpened by pressing one of two keyboard buttons, and each trial was initiated with a key-press. Within a block, 11 levels of image clarity ranging from blurred to sharpened (slope indices between −0.5 and +0.5 in steps of 0.1) were shown 20 times each, in pseudo-random order, for a total of 220 trials. Images consisted of 11 different natural scenes (landscapes) that were filtered at each of the 11 levels of image clarity.

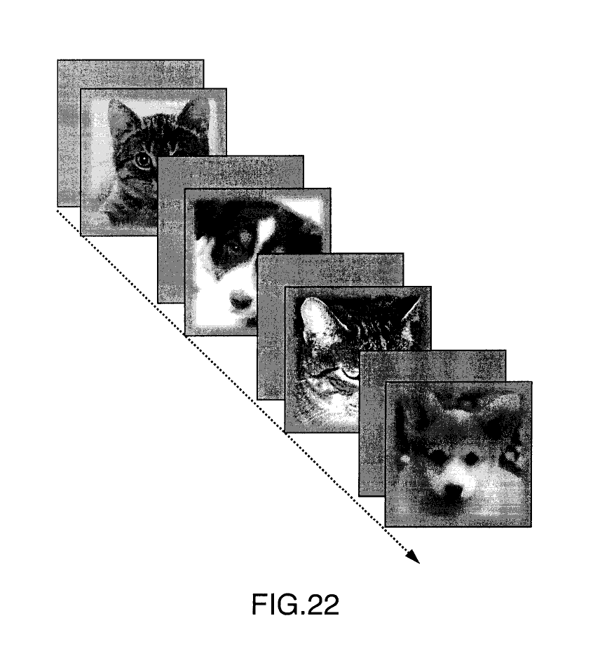

Adaptation. To evaluate the effect of adaptation on image clarity, the adaptation condition was similar to the baseline block, but included adapting images. An initial adapting image was presented for 1 minute at the beginning of the block, and was shown again for 5 s at the onset of each trial, as illustrated in FIG. 17. The same adapting image was used throughout a block, but differed from the test images. Adapting images had a slope index of ±0.5 (+adapt to sharpened image, −adapt to blurred image). As in the baseline conditions, test images consisted of 11 different natural scenes (landscapes) that were filtered at each of the 11 levels of image clarity. Each level was repeated 20 times, for a total of 220 trials per run.

Psychometric functions were fit to data from each block separately with a cumulative Gaussian function, the mean of which (50%) corresponds to the point of subjective equality. For all conditions, each block was repeated a minimum of 3 times per condition, to provide measures of variance, and the PSEs extracted from each run were averaged. Several testing sessions (4-6) were required to complete psychophysical experiments, as runs involving adaptation to sharpened images and to blurred images were tested in separate sessions. Furthermore, the baseline task was tested at the beginning of each session to avoid any adaptation-induced biases. Testing order for adaptation runs was randomized.

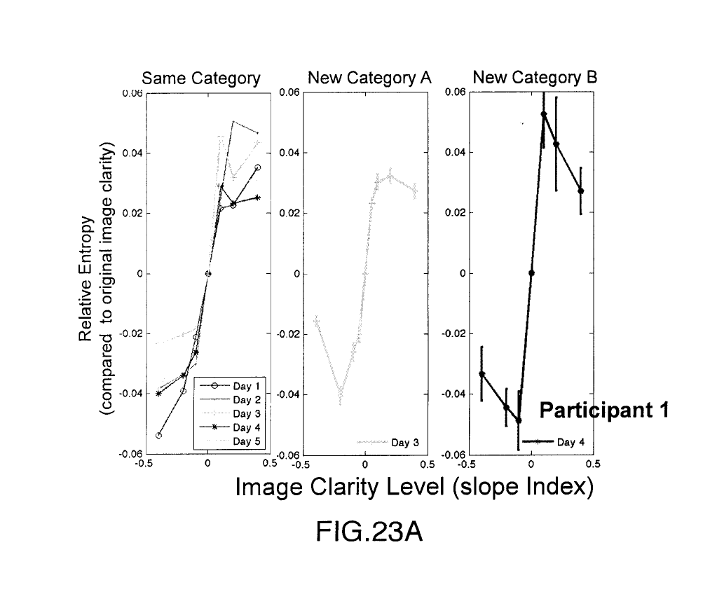

Physiological: Near-Infrared Spectroscopy. The optode array (3 by 11 formation, 52 channels) was placed at the back of the skull, with the bottom row centered above Oz, so that according to the 10/20 system, covered an area corresponding to occipito-temporal cortical regions as illustrated in FIG. 18. To evaluate the effects of adaptation to image clarity on physiological responses, two tasks were designed for NIRS measures.

Landscapes. A block design was used for this condition. Within an experimental run, 3 levels of image clarity were shown, with a slope index of 0 for the original image clarity, −0.5 for blurred, and +0.5 for sharpened. Each level of image clarity was blocked separately and contained 22 images that were presented in random order. Image exposure duration was 1500 ms, and images were separated by 2000 ms of mean gray background, for a total block duration of 77 s. Each block was repeated 4 times in pseudo-random order. In addition, a block consisting of gray background was interleaved between each of the image clarity blocks; its duration was 20 s, as a minimum of 16 s is required for the hemodynamic response function to return to baseline. Total run duration was 26 mn. To ascertain that observers attended to the task, they were required to indicate with a key press whether the image appeared blurred or sharpened, as they had in psychophysical runs.

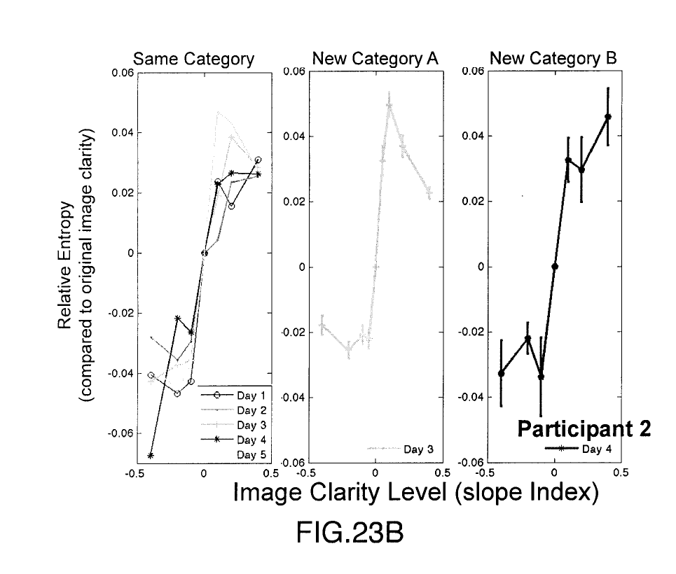

Birds/Mammals. In the landscapes task (above) responses were tied to the measure. Here, a bird-mammal classification was devised instead of one pertaining directly to image clarity (ie. blurred or sharpened), because directing attention away from the feature of interest (blur) allows for attention to be distributed equally across image clarity values. Within an experimental run, 3 levels of image clarity were shown, with a slope index of 0 for the original image clarity, −1 for blurred, and +1 for sharpened. Each level of image clarity was blocked separately and contained a total of 40 images (20 birds and 20 mammals in nature settings), and observers were required to specify by pressing one of two keyboard buttons whether the image contained a bird or a mammal. Images were presented for 1500 ms each, in random order and were separated by 2000 ms, so that block duration was 140 s, as illustrated in FIG. 19. Each block was repeated 4 times in pseudo-random order and preceded by a 20 s block of gray background, for a total run duration of 32 mn.

NIRS measures required 1 or 2 sessions to complete the experiments. Tasks were run in the same session, or spread across two sessions depending on participant comfort, availability, and preference.

Aberrometry. We chose to measure optical aberrations because they can influence perceived image clarity. Their effect on perceived image clarity was not assessed directly, but rather, the interaction between PSE shift and aberrations was established via correlations. Lower-order aberration measures (defocus and astigmatism) along with higher-order aberrations (3d, 4th, and 5th) were recorded. Aberrometry measures were repeated a minimum of three times per eye, and each measure was averaged across repetitions for each eye separately.

Results and Discussion

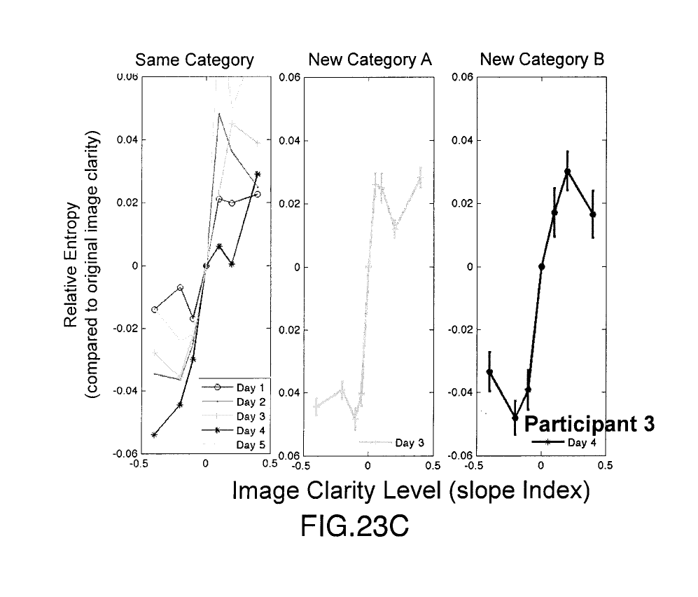

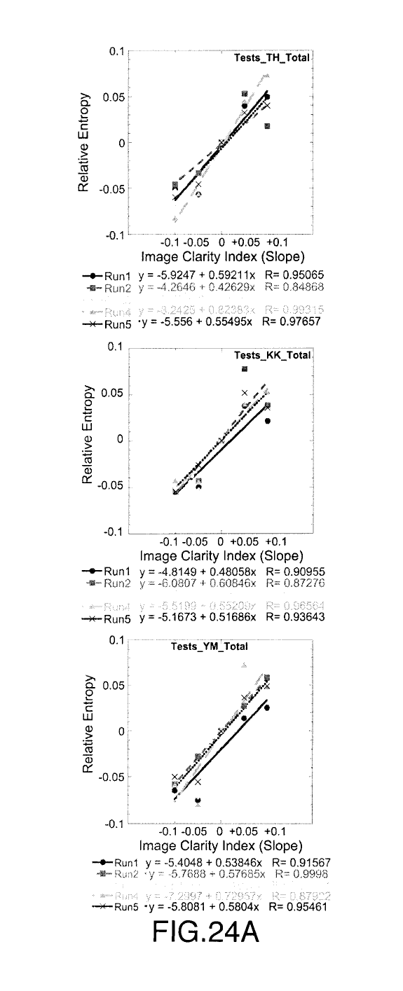

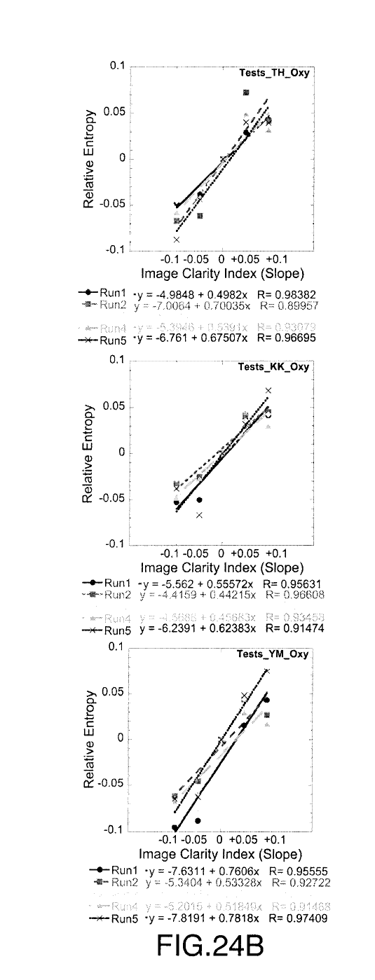

Behavioral: Psychophysics. The PSE was extracted on each run for each observer and averaged across runs, to provide a mean PSE for each condition as illustrated in FIGS. 20a and 20b, and Table 1. FIGS. 21a-21j show a slightly different average: individual plots where each image clarity level is averaged across runs and a cumulative Gaussian is fit through these average points (the PSE extracted from this fit through the averaged points was very close to the average PSE from separately-fit runs). This set of graphs permits to visualize individual patterns and consistency via error bars. Differences in PSE across conditions are significant for half the observers (s6, s7, s10, s11, and s13), where psychometric functions and PSEs tend to shift towards the left (negative values or blurrier images) after adaptation to blurred images and towards the right (positive values or sharpened images) after adaptation to sharpened images. Furthermore, approximately half of the observers display high sensitivity as evidenced by the steep functions (s10, s11, s13, s14), whereas the others did not. Average shifts in PSE for each observer, where the magnitude of the effect is characterized, are shown in the right-most columns of table 1 and illustrated in FIGS. 20a and 20b. Generally, observers display a shift in PSE (baseline, adapt blurred, adapt sharpened), as indicated by a main effect of condition (F[2,29]=25.36, p<0.0001). However, the shift arises largely from the difference between the baseline and adaptation to blur conditions (p=0.0002), and from the difference between adaptation to blur and adaptation to sharpened images (p<0.0001), but adapting to sharpened images does not yield a consistent shift in PSE from baseline (p=0.3). Though this pattern arises when averaging across subjects, it is consistent only in half the subjects, and as such, we focus primarily on individual data as opposed to group data.

TABLE 1
Psychophysics: mean Point of Subjective Equality (±1 SEM) for each observer for
each condition (baseline, adapt blur, adapt sharp), and differences (i.e. shift in PSE)

| | | | | | | | PSE shift | | | |
|---|---|---|---|---|---|---|---|---|---|---|
| | Condition | | | | | | adapt Blur − | | adapt Sharpen − | |
| Subject | Baseline | | Adapt Blur | | Adapt Sharp | | Baseline | | Baseline | |
| s6 | −0.048 | (0.105) | −0.187 | (0.099) * | 0.031 | (0.026) | −0.138 | (0.099) | 0.089 | (0.033) |
| s7 | 0.015 | (0.091) | −0.212 | (0.016) * | −0.056 | (0.081) | −0.227 | (0.016) | −0.070 | (0.081) |
| s8 | −0.080 | (0.062) | −0.178 | (0.231) | 0.104 | (0.070) | −0.098 | (0.231) | 0.185 | (0.070) |
| s9 | −0.319 | (0.033) | −0.423 | (0.073) | −0.360 | (0.052) | −0.104 | (0.073) | −0.041 | (0.052) |
| s10 | −0.070 | (0.029) | −0.173 | (0.022) *$ | 0.024 | (0.014) *$ | −0.103 | (0.022) | 0.094 | (0.014) |
| s11 | 0.003 | (0.053) | −0.125 | (0.041) *$ | 0.034 | (0.081) $ | −0.128 | (0.041) | 0.031 | (0.081) |
| s12 | −0.137 | (0.123) | −0.269 | (0.035) | −0.184 | (0.018) | −0.132 | (0.035) | −0.047 | (0.018) |
| s13 | −0.082 | (0.031) | −0.140 | (0.025) *$ | −0.020 | (0.025) *$ | −0.058 | (0.025) | 0.062 | (0.025) |
| s14 | −0.162 | (0.004) | −0.210 | (0.028) | −0.099 | (0.041) | −0.049 | (0.028) | 0.062 | (0.041) |
| s15 | −0.112 | (0.041) | −0.201 | (0.053) | −0.136 | (0.002) | −0.090 | (0.053) | −0.025 | (0.002) |

between each adaptation condition and the baseline (two rightmost columns, no statistics). Asterisks* indicate adaptation conditions that are significantly different from baseline, and dollar symbols$ indicate adaptation conditions that are significantly different from each other (adapt blurred vs. adapt sharpened) with p<0.05.

These adaptation effects are in general agreement with earlier work, but some differences appear. Participants were more sensitive to blur with respect to other work, presumably because of differences in observer expertise. Psychometric functions were not shown in previous works and cannot be compared to our findings. Furthermore, previous works report effects of both blurred- and sharpened-adaptation, whereas we do not show a consistent effect of sharpened adaptation (only 2 out of 10 observers). This could arise from a difference in the image clarity test levels used: theirs were confined to a narrower range than ours, or from a difference in observer experience. In accordance with our findings, a previous work tested a broader range of image clarity levels and a large number of participants, reported that adaptation effects were more variable and less symmetrical thank previous work.

Most of our observers' data were well fit by a cumulative Gaussian (r>0.97 in most instances, though subjects displaying high variability could have fits as low as r>0.7), and that fits did not improve with other functions. This likely arises from two facets: 1—high variability within a single subject's performance, and 2—that many observers do not display symmetric functions, but rather, functions that collapse with increasing sharpness. In other words, even though a highly sharpened image contains much detail, observers often rate it as being blurred, but a highly blurred image is rarely categorized as sharpened. We find this pattern in half of our observers (s7, s8, s11 (weak trend), s12, and s15). Interestingly, 4 of these observers are the ones with the most variable responses. (s7, s8, s11, and s12).

Physiological: NIRS. NIRS data indicate that image clarity elicits significant differences in hemodynamic response between conditions as illustrated in Table 2 which lists significance values between combinations of two conditions for each observer, separately for each of the two tasks. The three observers who show few/no significant interactions (s6, s10, s13) are the same three for whom signal strength from the array was weak, because they had thicker and longer hair. Otherwise, NIRS data show consistent differences between image clarity conditions, regardless of whether adaptation effects were significant behaviorally. For example, observers s8, s9, s12, s14, and s15 did not exhibit any significant adaptation effects psychophysically, and yet, all show consistent effects of adaptation to image clarity condition in NIRS; this even holds for observer 8, whose psychophysical data was extremely noisy.

TABLE 2
NIRS measures: p values for contrasts between conditions for each observer for
each of the two tasks. Asterisks* indicate significant contrasts with p < 0.02. Note that
observers s6, s10, and s13 all displayed weak signal strength when testing the optode
array.

| | Landscapes (sharpened or blurred?) | | | | Mammals or Birds? | | | |
|---|---|---|---|---|---|---|---|---|
| | original/ | original/ | original/ | blurred/ | original/ | original/ | original/ | blurred/ |
| Subject | gray | blurred | sharpen | sharpen | gray | blurred | sharpen | sharpen |
| s6 | 0.0057* | 0.0620 | 0.2887 | 0.0012* | 0.4578 | 0.1249 | 0.0156* | 0.6624 |
| s7 | 0.3506 | 0.0000* | 0.0000* | 0.9827 | | | | |
| s8 | 0.0000* | 0.0001* | 0.0000* | 0.0813 | 0.8608 | 0.0084* | 0.0018* | 0.0184* |
| s9 | 0.0148* | 0.0182* | 0.0166* | 0.5179 | 0.7904 | 0.0285* | 0.0002* | 0.0000* |
| s10 | 0.1260 | 0.4930 | 0.0141* | 0.1160 | 0.0051* | 0.0007* | 0.6805 | 0.0564 |
| s11 | 0.0000* | 0.1144 | 0.0000* | 0.0000* | 0.8360 | 0.1334 | 0.0219* | 0.0006* |
| s12 | 0.0034* | 0.0000* | 0.0000* | 0.1538 | 0.0000* | 0.0000* | 0.0000* | 0.0000* |
| s13 | 0.9770 | 0.8949 | 0.8841 | 0.7539 | 0.0051* | 0.7854 | 0.2799 | 0.2515 |
| s14 | 0.0000* | 0.0000* | 0.0000* | 0.0228* | 0.0000* | 0.0000* | 0.0002* | 0.0000* |
| s15 | 0.0000* | 0.0000* | 0.0376 | 0.0000* | 0.0110* | 0.0055* | 0.0001* | 0.0344* |

Furthermore, correlations between aberrometry measures and adaptation, or acuity, yielded no significant interactions.

In summary, we find that psychophysical measures of image clarity can be highly variable between, and even within individuals (despite many repetitions), but that NIRS measures provide a consistent response across observers when the signal is sufficiently strong.

### EXPERIMENT 2

The goals of this study were to obtain an objective and reliable measure of adaptation to perceived image quality despite differences in participant response consistency. However, NIRS measures over occipital brain regions were not possible in observers with long thick hair, because the signal was not sufficiently clear.

Large scale cortical connections exist between occipital, temporal and frontal cortices. Brain regions lying behind the forehead correspond to prefrontal cortex, which is generally associated with executive function (decision-making, planning, etc.). These areas use sensory information for their processes (i.e. decision-making or other) but sensory differences are not usually extracted here. In assessing image clarity from frontal regions, it is essential that the variable of interest (image clarity) is not confounded by the categories on which the decision rests. For example, in the birds/mammals classification used in experiment 1, an equal number of birds and mammals were shown and each category contained the same number of blurred, sharpened or original images, so that image clarity and the categorization decision were orthogonal to each other. Furthermore, when attention is directed away from the feature of interest, here image clarity, it allows for attention to be distributed equally across image clarity values. For these reasons, the bird/mammal protocol is used here.

Methods

Participants

The same observers as in experiment 1 participated here, but only 7 of the 10 for whom we had complete data sets were able to return for the present measures (observers s9, s10, and s13 could not return).

Procedure

NIRS. The optode array (52 channels, in 3 by 11 formation) was placed on the forehead centered above the nasion, so that it covered an area corresponding to frontal cortical regions according to the 10-20 system. NIRS measures required a one-hour sessions to complete the experiments. Data from the 52 channels were compared statistically for each observer. Two-tailed t-tests were used to compare image clarity conditions for each subject, and the alpha level was corrected to p<0.02 to account for the number of image clarity conditions contrasted.

Results and Discussion

NIRS. NIRS data indicate that image clarity elicits significant differences in hemodynamic response when measuring over frontal brain regions, and that these differences are consistent with those obtained from occipito-temporal measures, as illustrated in Table 3 that lists significance values between combinations of two conditions for each observer, separately for each of the two measurement conditions. NIRS measures of image clarity over frontal regions yields similar patterns to those obtained from occipito-temporal regions. NIRS-measured adaptation over frontal regions yields consistent results despite behavioral variability (Experiment 1).

TABLE 3
NIRS measures: p values for contrasts between conditions for each observer for
each of the two tasks. Occipito-temporal measures are those described in the previous
report, and frontal measures are newly acquired. Asterisks* indicate significant
contrasts with p < 0.02. Note that observers s9, s10, and s13 were unable to return for
new measures.

| | Birds or Mammals: Occipital-Temporal | | | | Birds or Mammals: Frontal | | | |
|---|---|---|---|---|---|---|---|---|
| | original/ | original/ | original/ | blurred/ | original/ | original/ | original/ | blurred/ |
| Subject | gray | blurred | sharpen | sharpen | gray | blurred | sharpen | sharpen |
| s6 | 0.4578 | 0.1249 | 0.0156* | 0.6624 | 0.0080* | 0.0273 | 0.0123* | 0.0021* |
| s7 | | | | | 0.0134* | 0.2270* | 0.0251* | 0.0003* |
| s8 | 0.8608 | 0.0084* | 0.0018* | 0.0184* | 0.4585 | 0.0000* | 0.0052* | 0.6637 |
| s9 | 0.7904 | 0.0285* | 0.0002* | 0.0000* | | | | |
| s10 | 0.0051* | 0.0007* | 0.6805 | 0.0564 | | | | |
| s11 | 0.8360 | 0.1334 | 0.0219* | 0.0006* | 0.0001* | 0.0000* | 0.0004* | 0.0000* |
| s12 | 0.0000* | 0.0000* | 0.0000* | 0.0000* | 0.0133* | 0.0001* | 0.2271 | 0.0000* |
| s13 | 0.0051* | 0.7854 | 0.2799 | 0.2515 | | | | |
| s14 | 0.0000* | 0.0000* | 0.0002* | 0.0000* | 0.0056* | 0.0000* | 0.6272 | 0.0000* |
| s15 | 0.0110* | 0.0055* | 0.0001* | 0.0344* | 0.0001* | 0.0000* | 0.0050* | 0.0000* |

### Experiment 3

### Introduction

Psychophysical measures of adaptation to image clarity were highly variable between, and even within individuals. In contrast, NIRS measures of adaptation to image clarity provided a consistent response across observers when measured over occipito-temporal brain regions, and interestingly, over frontal regions also. However, these differences were restricted to coarse comparisons between image clarity: one level each of blurred and sharpened and one original. Psychophysical baseline measures of perceived image clarity indicated that observers were sensitive to subtle changes. In order to establish whether NIRS measures could reflect this sensitivity images that were only slightly blurred or slightly sharpened were presented in rapid interleaved order (event-related design, no adaptation). Furthermore, one of our experimental goals was to measure perceived image clarity in a broadly applicable context. So far, our NIRS measures required long blocks of continuous testing. In order to reduce the testing length needed to obtain significant results, we ran separate runs of 10 minutes four times.

Methods

Participants

Three of the same observers who had participated in experiments 1 and 2 returned to participate in these experiments (s7, s14, and s15), and an additional 4 observers were recruited, for a total of 7 observers.

Procedure

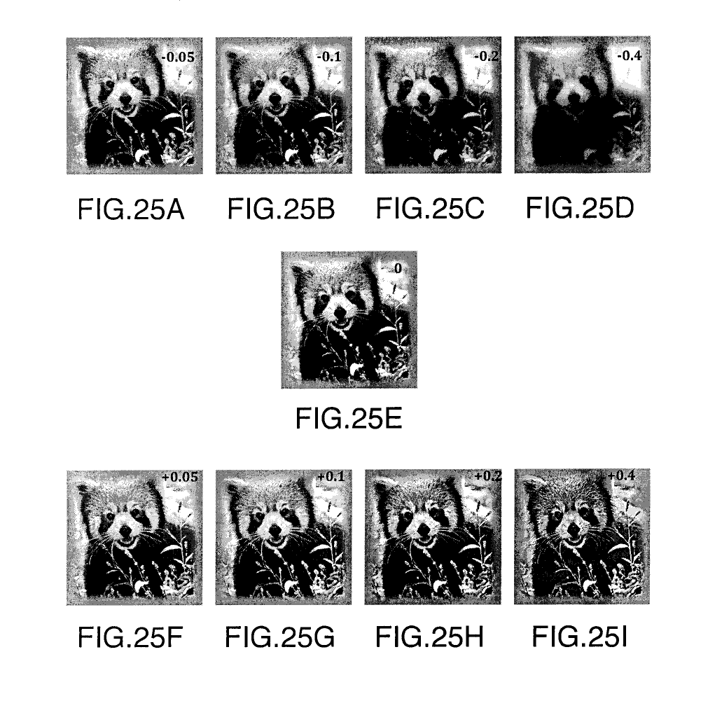

The optode array (24 channels, in 3×6 formation) was placed on the forehead, using the 10-20 system, so that it covered an area corresponding to frontal cortex. In order to continue using a categorization that was unrelated/orthogonal to the variable of interest (not confounded), while maintaining participant interest, we devised a similar task using a cat or dog classification. As such, observers were required to specify by pressing one of two keyboard buttons whether the image contained a cat or a dog. Within an experimental run, 7 levels of image clarity were shown: slope indices of 0 for original image clarity, −0.1, −0.2, −0.4 for blurred, and +0.1, +0.2, and +0.4 for sharpened. Each image clarity level contained a total of 20 images (10 cats and 10 dogs in nature settings), and one image from each clarity level was presented in pseudo random order, for a total of 140 trials per run, during which no single image was repeated twice. Images were presented for 1000 ms each in random order and the next image appeared 2000 ms after observers pressed a key with their response, as illustrated in FIG. 22. Total testing time was approximately 10 mn per run. Each run was repeated 4 times.

The levels of blur and sharpening were selected to sample the most dynamic part of the psychophysical function and one near-saturation, for most observers, based on behavioural measures described in earlier reports. All new observers participated in the baseline psychophysical task only. Briefly, the baseline task consisted of deciding whether a briefly presented image looked blurred or sharpened (too detailed) for several subtle levels of image clarity ranging from highly blurred, to highly sharpened, and going through 0 or the original “clear” image. The image clarity value corresponding to the point at which observers respond at 50%, corresponds to the point of subjective equality or “clear” image representation.

Analysis

For all NIRS measures, results were analyzed for 24 channels from the 1-second stimulus presentation window. They were averaged for each level of the same image clarity (as there is no block to average over). All other analyses were identical to the block design.

Results and Discussion

NIRS data indicate that subtle differences in image clarity elicit significant changes in hemodynamic response when using an event-related design in most observers (all but s17) as illustrated in Tables 4a and 4b. NIRS data showed consistent differences between image clarity conditions. The two smallest levels of blur and sharpening (ie. blur1/blur2 and sharp1/sharp2) were not clearly differentiated, but each was strongly distinct from the original image (blur1/original and sharpened1/original). This suggests that a robust separation exists between the original (best) image clarity and the mildest amounts of image manipulation.

TABLE 4a
ANOVA results for each subject. Repeated Measures ANOVA tables for
Channel (24) × Levels of image Clarity (7). When there was a significant
main effect of level and/or a significant interaction between level and
channel, corrected t-tests described in table 1 were conducted to assess
differences between original image clarity and all other levels of image
clarity. For cases where the interaction but not the main effect of level
was significant, we proceeded to the planned comparisons, because the
effect of level could be washed out by the dramatic effect of channel.

| | | ANOVA statistics | | |
|---|---|---|---|---|
| Subject | Variables | F | p | df |
| S7 | Channel | 2471 | .000 | 23 |
| | Channel × Level | 5.009 | .000 | 138 |
| | Level | 1.95 | .06 | 6 |
| S14 | Channel | 3225 | .000 | 23 |
| | Channel × Level | 2.915 | .000 | 138 |
| | Level | 1.59 | .01 | 6 |
| S15 | Channel | 5899 | .000 | 23 |
| | Channel × Level | 8.5 | .000 | 138 |
| | Level | 6.4 | .000 | 6 |
| S17 | Channel | 3035 | .000 | 23 |
| | Channel × Level | 6.6 | .000 | 138 |
| | Level | 5 | .000 | 6 |
| S18 | Channel | 15105 | .000 | 23 |
| | Channel × Level | 2.496 | .000 | 138 |
| | Level | .34 | .9 | 6 |
| S19 | Channel | 11577 | .000 | 23 |
| | Channel × Level | 2.8 | .000 | 138 |
| | Level | 3.628 | .001 | 6 |

TABLE 4b
t-test comparisons. p values for contrasts between levels of image clarity
(original and varying levels of blur) for each observer. The run number in the right-most
column indicates the run from which significant effects arose; runs in parenthesis
yielded weaker effects than values listed in the table. Asterisks indicate significant
contrasts with *** at p < 0.0001, ** at p ≦ 0.001, * at p ≦ 0.008. Once more, maximum
threshold for significance (p-value) varies previous tables, because of correction factors
for the number of alternatives compared.
Cats/Dogs Event-related Design: No Adaptation
Frontal measures of Subtle Differences in Image Clarity

| | Contrasts with Original images | | | | | | between blur levels | | | Between sharp levels | | | |
|---|---|---|---|---|---|---|---|---|---|---|---|---|---|
| Subj | o/b1 | o/b2 | o/b4 | o/s1 | o/s2 | o/s4 | b1/b2 | b1/b4 | b2/b4 | s1/s2 | S1/s4 | s2/s4 | run |
| s7 | *** | >0.1 | ** | *** | 0.04 | *** | ** | >0.1 | *** | >0.1 | *** | *** | (1), 2 |
| s14 | *** | *** | >0.1 | *** | *** | >0.1 | >0.1 | *** | *** | >0.1 | *** | *** | 1 |
| s15 | ** | ** | * | * | * | * | >0.1 | * | * | >0.1 | >0.1 | >0.1 | 2, 3, 4, avg |
| s17 | >0.1 | >0.1 | >0.1 | >0.1 | >0.1 | >0.1 | >0.1 | >0.1 | >0.1 | >0.1 | >0.1 | >0.1 | — |
| s18 | * | >0.1 | * | * | >0.1 | * | *** | ** | 0.04 | ** | * | .014 | 1, (2) |
| s19 | *** | *** | *** | *** | *** | * | *** | *** | ≧0.1 | *** | *** | *** | 1, 2 (3, 4) |

The right-most column indicates the run number for which significant effects were found. Averaging across runs weakened or even abolished any significant findings. This is not surprising given that significant effects tended to appear in earlier runs (1 or 2), as opposed to later runs where effects often disappeared. This likely arose because of increasing observer familiarity with the images and task, as previous work has shown that hemodynamic responses decrease with repetitions of a same stimulus (also known as hemodynamic response-adaptation).

In addition, participants could attend differently to images of varying quality, in which case signal strength would vary between levels of image clarity. As such, signal-to-noise ratios (SNR) were evaluated for each level of image clarity and were found to be similar across levels. This suggests that the significant effects we report here did not arise from varying signal strength, but rather, from differences in hemodynamic response patterns.

General Discussion and Conclusions

Our findings indicate that behavioral measures of perceived image clarity for the baseline condition (no adaptation) are robust and provide information regarding individual bias towards blur. In contrast, adaptation measures of shifts in perceived image clarity are variable and inconsistent. Half of our observers exhibited shifts induced by adaptation to blurred images, of which only two displayed a weak effect of adaptation to sharpened images; adaptation effects could not be measured in the remaining half. Despite such inconsistent behavioral results, NIRS measures of adaptation to blurred, sharpened and original images yield consistent differences between these conditions. These findings hold for NIRS measures over occipital, and even frontal cortical regions. Furthermore, NIRS over frontal cortex is sensitive to subtle differences in image clarity without adaptation and can be assessed in as little as 10 mn.

Taken together, these findings suggest that physiological measures can yield objective measures of adaptation state regardless of individual variability in performance. Interestingly, responses to detailed visual features normally associated with occipital function, can be extracted from frontal measures. This suggests that occipito-frontal connections convey information that is not only relevant to the decisional process, but also to perceived stimulus quality.

In the following further experimental results are provided with respect to the visual clarity assessment.

Repeatability

When testing the image-clarity NIRS protocol that yielded significant results, the same about 10 mn tests were repeated four times (i.e. four runs) during a same testing session. This protocol had been adopted, because most brain imaging techniques require as many repetitions as possible to yield significant results. We found that such extensive repetition was not necessary for the present protocol, and the results were most significant for the first and/or second runs. The reason for weakened results in later runs was likely related to subjects learning the task and habituating to it. The functional MRI imaging literature has shown that maintaining a constant level of attention allows for better hemodynamic response and therefore cleaner results.

The purpose of the present experiments was to run the tests over 5 days using different categories.

Participants (n=3) were tested over numerous sessions (5) on different days.

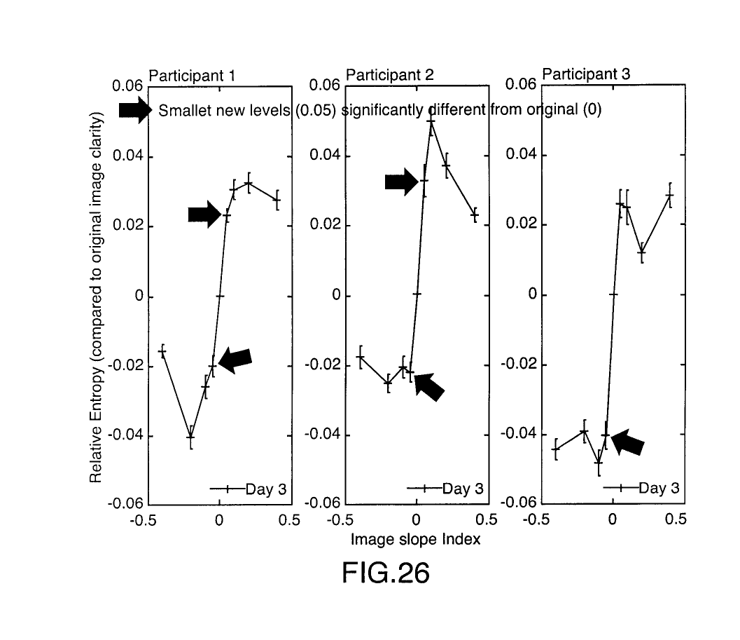

Each session consisted of 2 NIRS runs: the first one was identical across sessions, and the second one varied from session to session. Results indicate that significant measures are obtained for repetition during a session over 2 runs, and from session-to-session, as illustrated in FIGS. 23a-c which each presents, for a respective participant, the relative entropy as a function of the image clarity level for a repetition of a same condition (cats and dogs) over 5 sessions, each tested on different days, for a new category A tested on day 3 and another new category B tested on day 4. The relative entropy plotted as a function of image clarity level was determined using basic state function analysis tools.

In the following, results for numerous runs during a single session are presented.

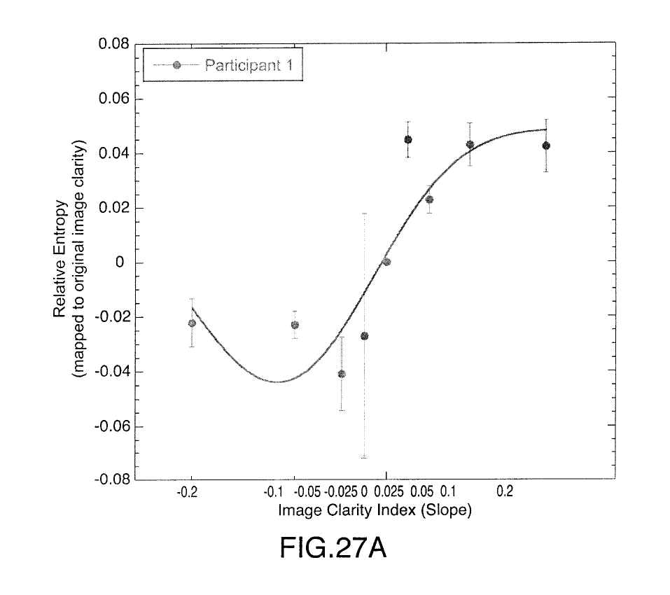

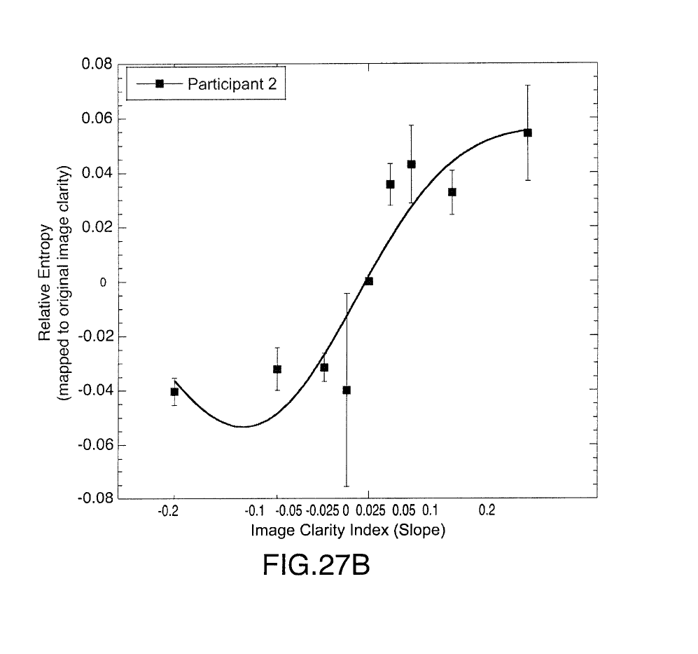

Participants (n=3) were tested on numerous tests (runs≧5) during a single session. Each session consisted of 5 NIRS runs, each from a different category. Results indicate that significant measures are obtained for numerous runs (5) during one session, as illustrated in FIGS. 24a and 24b which each present the relative entropy as a function of the image clarity level with linear fits and slope estimates. Repeatability is thus confirmed. In FIG. 24a, the relative entropy was determined from the oxy-hemoglobin while the relative entropy was determined using the total hemoglobin in the case of FIG. 24b.

Sensitivity

We had already shown measurable differences for subtle changes in image clarity using NIRS, but measuring sensitivity to extremely fine differences would prove even more powerful. Our measures thus far, included 7 levels of image clarity (3 blur, 3 sharpened, and one original), but we were interested in an even finer scale.

The purpose of the present study was to assess sensitivity to test a finer scale of image clarity, by introducing levels that were even closer to the original than those already used.

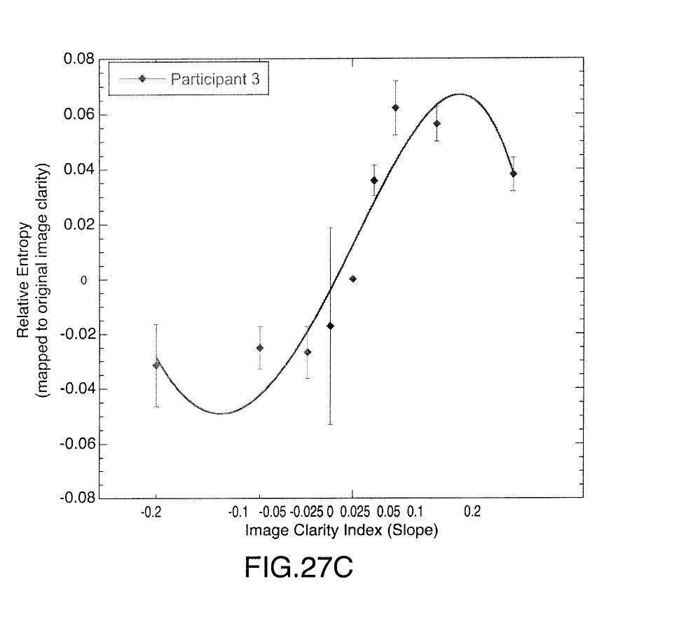

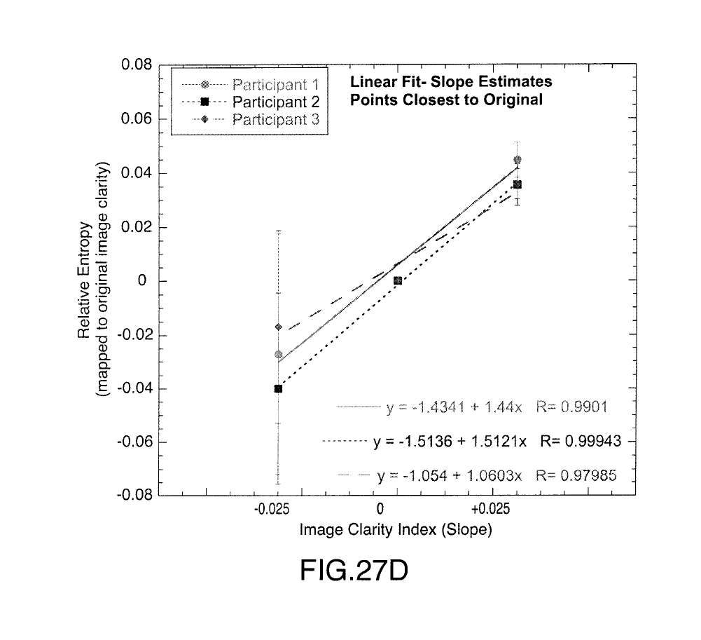

In the above-presented experimental results, we used 7 levels of image clarity (−0.4, −0.2, −0.1, 0, +0.1, +0.2, +0.4; −blur, +sharpen, 0 original). We have now tested NIRS-measures of perceived clarity over 9 levels, with the two new levels of +0.5 added at the finest end of the scale: (−0.4, −0.2, −0.1, −0.05, 0, +0.05, +0.1, +0.2, +0.4; FIG. 4.2-levels). FIGS. 25a-i present the images having the different levels of image clarity. This scale yielded significant differences in NIRS measures of perceived image clarity as illustrated in FIGS. 26a-c which each present, for a respective participant, the relative entropy as a function of the image clarity level.

For the present NIRS testing, we present images on a computer screen, and the computer sends a signal, or marker, to the NIRS device in order to indicate the image clarity level that is presented. We have different markers for each of the levels of image clarity. Current analyses and figures make relative comparisons between the original image and all other image clarity levels. The next consideration is to make relative comparisons for each of the levels across all conditions (e.g. blur levels compared to other blur levels). Results indicate that NIRS measures are sensitive to the weakest levels of image clarity change from original. Other levels also differed significantly from the original image clarity.

In the above, we had already shown measurable differences for subtle changes in image clarity using NIRS, but measuring sensitivity to extremely fine differences would prove even more powerful. Our measures included 7 levels of image clarity (3 blur, 3 sharpened, and one original, specifically, (−0.4, −0.2, −0.1, 0, +0.1, +0.2, +0.4; −blur, +sharpen, 0 original), and also 9 levels, with ±0.05 added at the finest end of the scale: (−0.4, −0.2, −0.1, −0.05, 0, +0.05, +0.1, +0.2, +0.4). In the following, we were interested in an even finer scale, where ±0.025 are added.

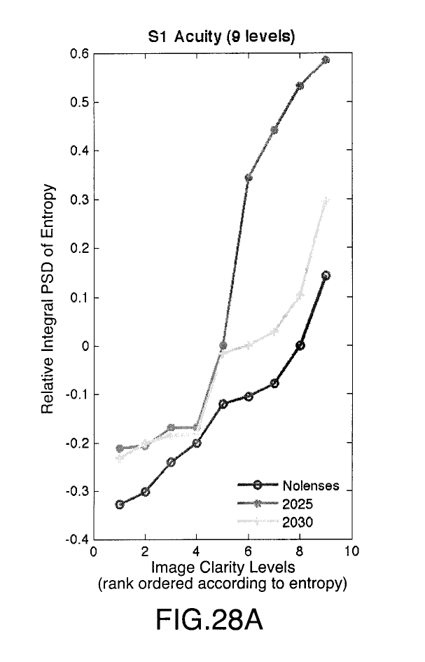

We have now tested NIRS-measures of perceived clarity over 9 levels, with new levels of ±0.025 added at the finest end of the scale. The 9 levels of clarity consisted of: −0.2, −0.1, −0.05, −0.025, 0, −0.025, +0.05, +0.1, +0.2. FIGS. 27a-c illustrates, for a respective participant, the relative entropy as a function of the image clarity level. FIG. 27d illustrates the relative entropy with linear fits and slope estimates on points closest to original for the same 3 participants. All data were based on a single run.

Acuity

In one embodiment, NIRS measures of perceived image clarity would vary when testing an individual with and without correction. Small changes in correction can induce small changes in acuity and different NIRS-measured profiles are expected.

The purpose of the present study is to evaluate sensitivity of the present NIRS-measured protocol for weak changes in acuity.

We tested 3 participants with 3 different acuities at a single viewing distance (114 cm, as for all of our testing). Each participant was tested at their best acuity (better than 20/20); positive sphere lenses were added to reduce their acuity to the 20/20-20/25 range, and to 20/30. The poorest acuity was tested first in a session, followed by the middle, and finally the best. Our finest testing scale was used, with 9 levels of image clarity (original, ±0.05, ±0.1, ±0.2, ±0.4). We find differences in NIRS-measured profiles for the various acuities and are thus able to characterize the profile corresponding to the best acuity. Sliding state function analysis was used to determine the entropy.

NIRS measures of our protocol in 2 participants (tested with 9 levels of image clarity), at 1 distance (114 cm) with three different acuities:

1—participant's best acuity: 20/15 (hollow circles);

2—acuity degraded to 20/20-20/25 (solid circles); and

3—acuity degraded to 20/30 (crosses)

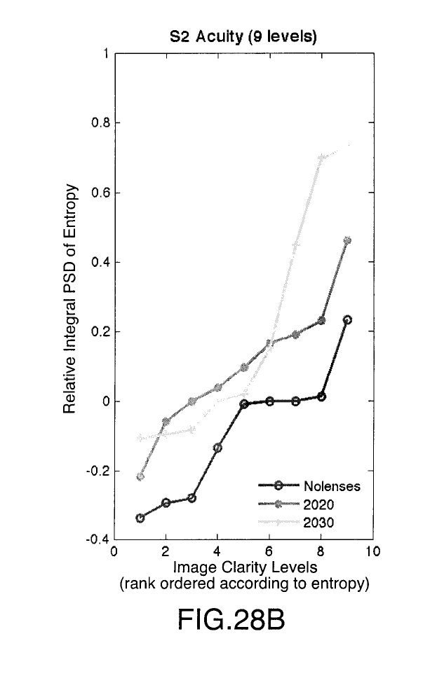

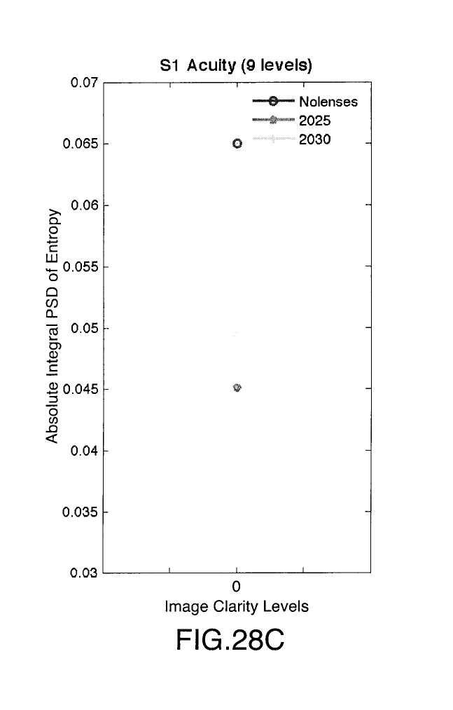

FIGS. 28a and 28b each present, for a respective participant, the relative integral of the entropy's power spectrum density as a function of the image clarity level, when the levels are rank-ordered by entropy in order to illustrate the entropy scatter for the overall function.

FIGS. 28c and 28d each present, for a respective participant, the absolute integral of the entropy's power spectrum density for the original image only.

To determine the clearest perception for each participant, the absolute entropy for the original image only is compared across acuity conditions. The point with the highest entropy indicates the condition with the best perceived clarity. Results for NIRS measures of perceived image-clarity with our windowed state function analysis indicate that the best perceived image clarity corresponds to the 20/15 acuity condition (hollow circle).

In the above-presented results, we showed that NIRS measures of perceived image clarity vary when testing an individual with and without correction. Small changes in correction induced small changes in acuity and different NIRS-measured profiles. The purpose of the below study is to evaluate sensitivity of the present NIRS-measured protocol for weak changes in correction.

Three participants were tested with several different corrections at a single viewing distance of about 55 cm. 9 levels of image clarity were used (original, +0.05, +0.1, +0.2, ±0.4). Two sets of prescription shifts were used: Set 1: +1.00, +0.50, +0.25, 0, −0.25, −0.5 and Set 2: +2.00, +1.50, +1.00, +0.50, 0. All conditions of each set were tested in a single session.

We analyzed the data using windowed state function tools and found differences in NIRS-measured profiles for the various corrections. In addition, our analysis was elaborated further, to identify best perceived image clarity and results were simplified so that graphs show a single point for each dioptric change instead of a full function.

Finally, some very weak changes in correction were used, that in some cases would not induce significant changes in acuity, and we were able to identify differences in the NIRS profiles.

In the following, there is presented NIRS measures in 3 participants (tested with 9 levels of image clarity), at a single distance (55 cm) for varying ocular correction shifts. The data were analyzed with windowed state function analysis on total-hemoglobin only.

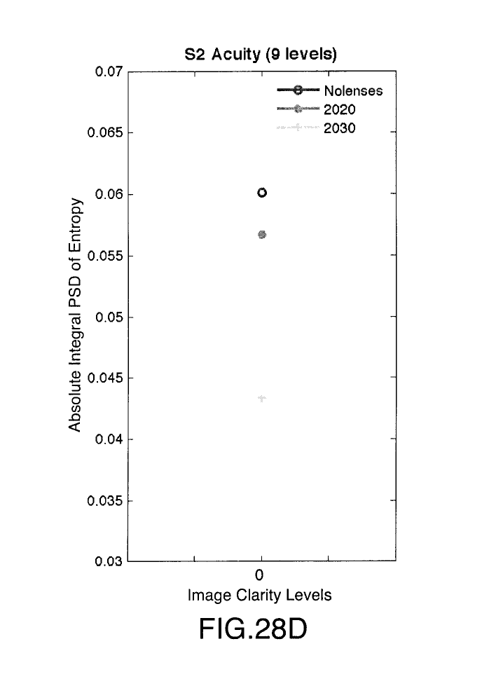

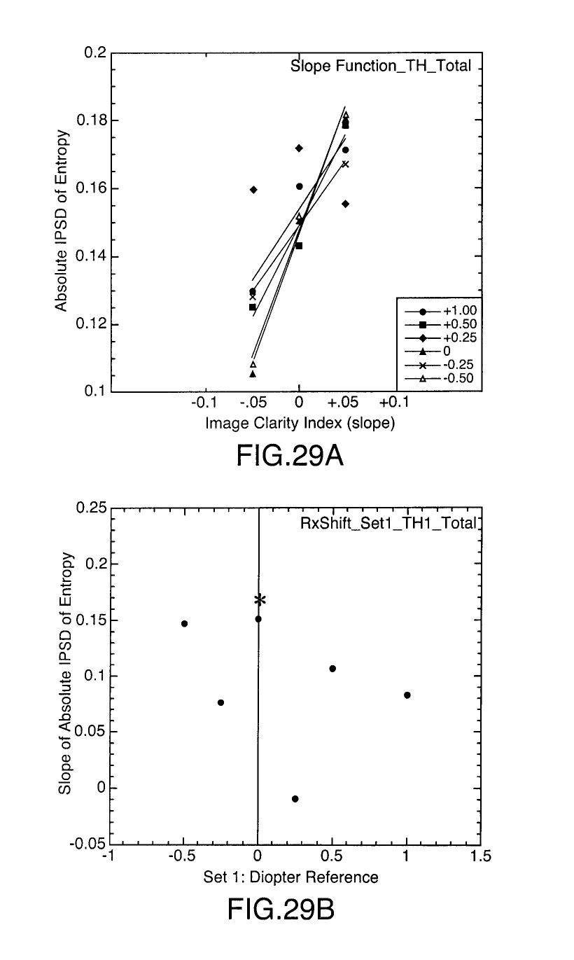

FIG. 29a presents the absolute integral of power spectrum density (IPSD) as a function of image clarity for each prescription shift with linear fit. Plotted for image clarity values closest to 0 and slope of linear fit values extracted. If a slope is negative, this indicates a bad run (i.e. NIRS measures were not good).

FIG. 29b presents the slope of the linear fit estimated in FIG. 29a, which is plotted for each Rx shift. The clearest perception of image clarity is indicated by highest value in this graph and marked by an asterisk (*).

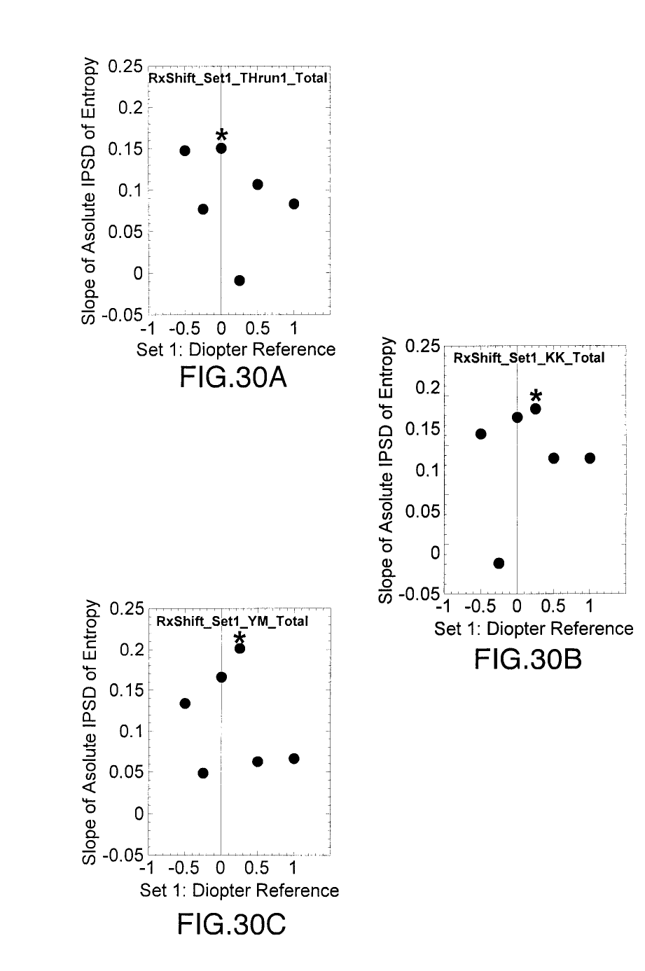

FIGS. 30a-c each presents the slope of the absolute IPSD as a function of the image clarity level for three different participants and for a first prescription shift set. The clearest perception of image clarity is given by the highest value indicated with an asterisk (*). For each participant, best diopter reference is:

Participant 1: diopter reference=0

Participant 2: diopter reference=+0.25

Participant 3: diopter reference=+0.25

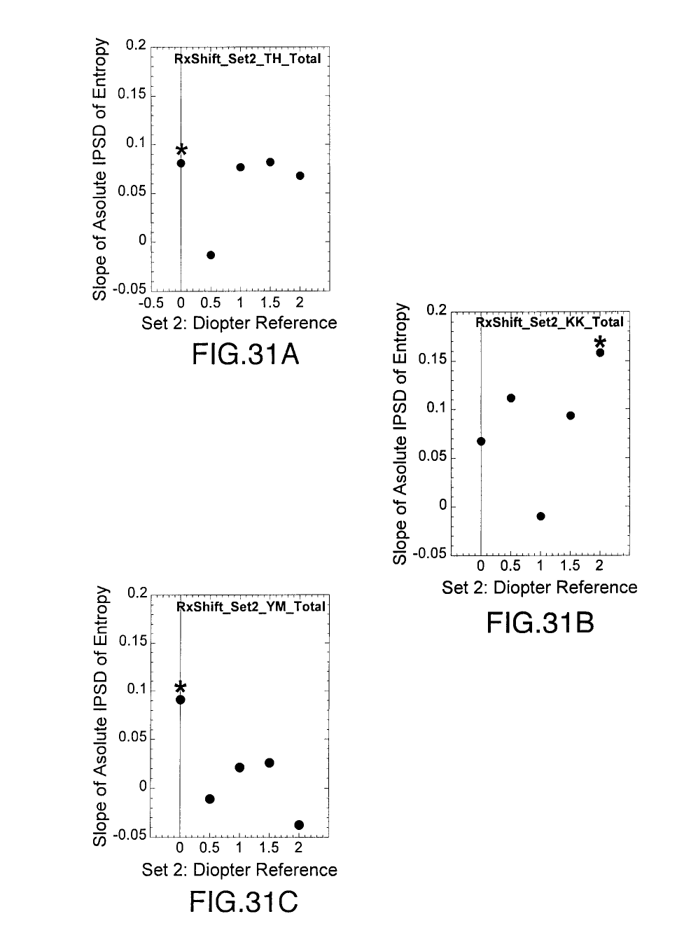

FIGS. 31a-c each presents the slope of the absolute IPSD as a function of the image clarity level for three different participants and for a second prescription shift set.

The clearest perception of image clarity is given by the highest value indicated with an asterisk (*). For each participant, best diopter reference is:

Participant 1: diopter reference=0;

Participant 2: diopter reference=+2.00; and

Participant 3: diopter reference=0.

Repeatability Across days of Prescription Shift Set 1

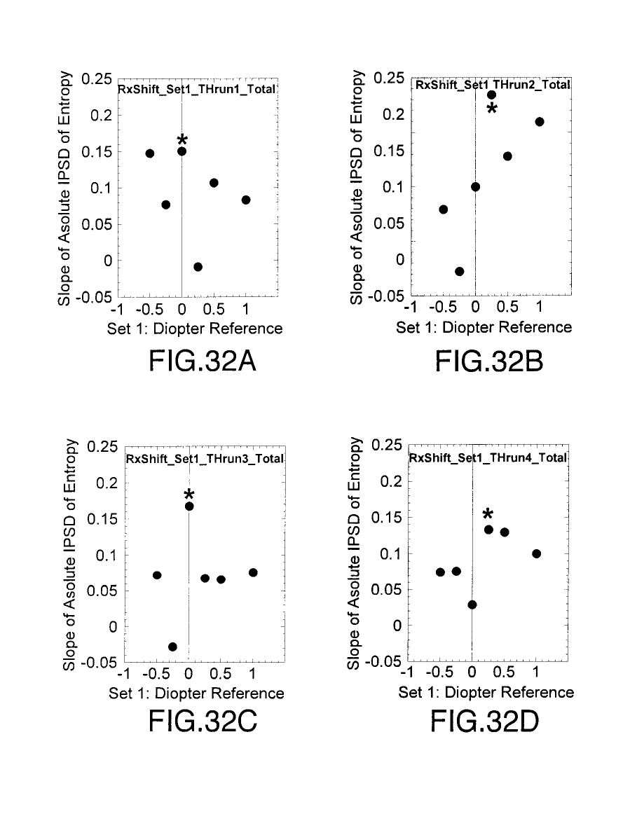

FIG. 32a-d present the slope of the absolute IPSD as a function of the image clarity level for a same different participant and for four different days. The clearest perception of image clarity is indicated by highest value* for participant TH. For each day, the best diopter reference is the following:

run 1: diopter reference=0;

run 2: diopter reference=+0.25;

run 3: diopter reference=0; and

run 4: diopter reference=+0.25.

Therefore, the best diopter prescription is between the current one, and the current one plus 0.25.

While in the above experimental results, the time duration of a test is about 10 min, it should be understood that the time duration may vary. For example, the time duration of a test may be greater than 10 min. In another example, the time duration of a test may be about 8 min, about 7 min, about 3 min, about 2 min, etc.

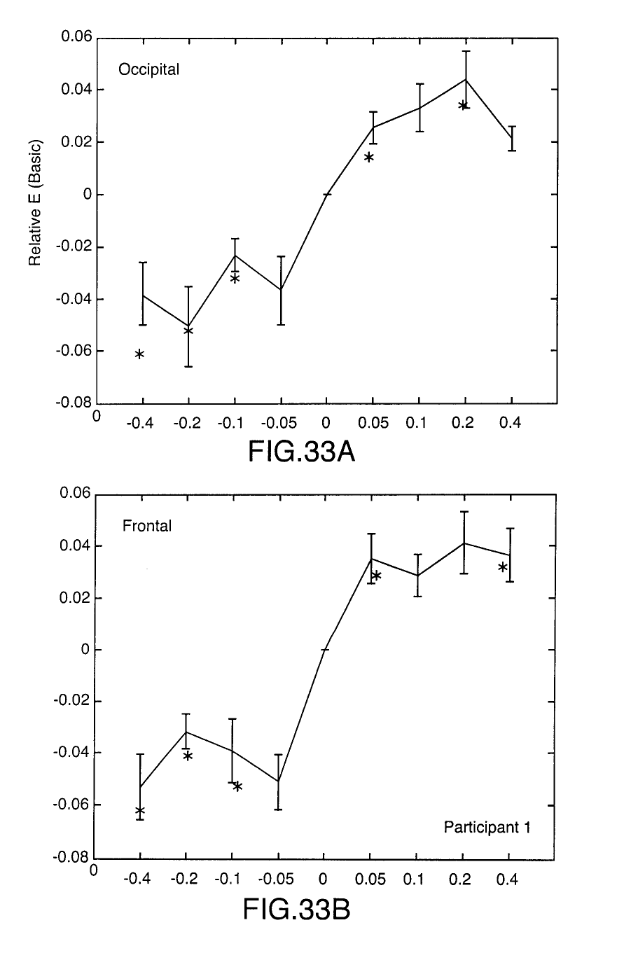

While in the above experimental results, the brain activity has been measured in at least one region of the frontal cortex, the following experimental results show that a stimulus characteristic may be assessed by measuring the activity of the region located at the frontier of the occipital cortex, the temporal cortex, and the parietal cortex. FIGS. 33a and 33b present the relative entropy as a function of an image clarity when several images having different clarities are presented to a same subject. FIG. 33a represents the relative entropy corresponding to the activity of the occipital cortex while FIG. 33b illustrates the relative entropy corresponding to the activity of the frontal cortex. Since the two entropy curves are similar, it may be concluded that it is possible to assess the perception of an image clarity.

While experimental results have been provided for the characterization of perceived visual clarity and prove the efficiency of the above described method 10 and system 20 in the case of visual clarity, the person skilled in the art would soundly predict that the method 10 and 20 would be efficient for characterizing the perceived clarity of sensory stimuli other than visual stimuli. Sensory systems operate according to similar principles and can influence each other. Furthermore, sensory stimuli can be described along identical dimensions that a human brain interprets, such as vibrations and spectra: examples exist for various senses including vision, audition, touch, and olfaction. A human brain interprets information from our senses in a similar way, and therefore, the above-described procedure with respect to vision is applicable to other senses.

In the following, experimental results are presented in order to show the perceptual equivalence between the image slope blur and the dioptric blur for weak changes from original image clarity. The use of slope manipulations to adjust image clarity is important for tests and data analysis protocol. However, its relation to real-world changes in dioptric power may be difficult to understand. The purpose of the experiments is to compare, perceptually (no physiological measurement), similarity between the perceived image clarity arising from changes in image slope and the perceived image clarity arising from changes in dioptric blur.

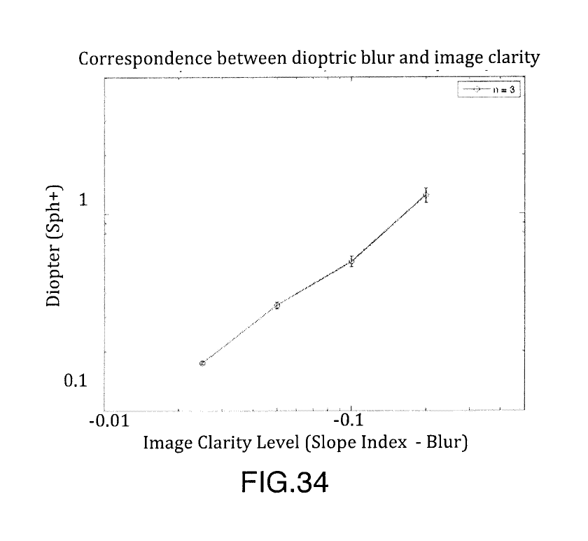

We asked 3 participants to match various levels of image clarity in digitally-blurred images (slope change) to optical-blur (diopter change). We found perceptual equivalence between +Dioptric blur and digitally blurred images (slope change), ranging up to a slope index of −0.2. Beyond this value, the two types of blur were qualitatively different and participants could not match the two. Image slope values of −0.025, −0.5, −0.1, and −0.2 were used. Comparisons were conducted from a viewing distance of 114 cm, and accommodation was accounted for. FIG. 34 illustrates the dioptric blur (+sphere) required to match perceived blur on image clarity slope scale for 3 participants.

While in the above description, the method and system are used for assessing the clarity of a stimulus perceived by a subject, it should be understood that the same method and system may be used for assessing the perception of stimulus properties other than clarity. Taking the example of a visual stimulus such as an image, the above method and system are used for assessing the perceived visual clarity of a subject. It should be understood that the perception of other image properties may also be assessed using the same method and system. For example, the subject's perception of a luminance contrast, a texture contrast, and/or noise in an image may also be assessed. In this case, images having different luminance contrast, different texture contrast, or different noise are presented to the subject who is asked to identify a characteristic that is not related to the luminance, texture or noise, respectively, for each image. By measuring the cerebral activity of the subject, it is possible to assess the perceived luminance contrast, texture contrast, or noise.

For example, a luminance-modulated image may be generated by the addition of an envelope (signal) with a carrier (texture) and texture-modulated images as their multiplication. For luminance-modulated images, the local luminance average varies throughout the image according to the envelope while the local contrast remains constant. For texture-modulated images, the local luminance average remains constant and the local contrast varies throughout the image according to the envelope. Therefore, because a Fourier transform can directly detect the signal frequency of luminance-modulated images, this type of stimulus is typically characterized as Fourier, first order, or linear. However, texture-modulated images are not considered as Fourier stimuli because the signal frequency is not present in the Fourier domain. Therefore, texture-modulated stimuli are characterized to be non-Fourier, second order, or nonlinear stimuli.

The person skilled in the art will understand the perception of first order characteristics, second-order characteristics, or noise may be assessed for stimuli other than visual, such as auditory, tactile, olfactory, or gustatory stimuli.

The embodiments of the invention described above are intended to be exemplary only. The scope of the invention is therefore intended to be limited solely by the scope of the appended claims.

1. A method for assessing a subject perception of a stimulus property, comprising:
applying to a subject at least a first sensory stimulus having a first value for the stimulus property and a second sensory stimulus having a second value for the stimulus property, the second value being different from the first value;
requesting the subject to identify a characteristic of the first and second sensory stimuli during the application thereof, the characteristic being unrelated to the stimulus property to be assessed, thereby focusing an attention of the subject on a decision making task;
measuring an activity of at least one brain region of the subject during the application of the first and second sensorial stimuli and the identification of the characteristic thereof, thereby obtaining a brain activity measurement; and
determining a difference of stimulus property perceived by the subject between the first and second sensory stimuli from the brain activity measurement, thereby characterizing the subject perception of the stimulus property.

2. The method of claim 1, wherein the stimulus property comprises a stimulus clarity.

3. The method of claim 1, wherein the stimulus property comprises one of a contrast and a noise.

4. The method of claim 1, wherein the first sensory stimulus and the second sensory stimulus each comprise a visual stimulus.

5. The method of claim 1, wherein the first sensory stimulus and the second sensory stimulus each comprise one of an auditory stimulus and a tactile stimulus.

6. The method of claim 1, wherein the first sensory stimulus and the second sensory stimulus each comprise one of an olfactory stimulus and a gustatory stimulus.

7. The method of claim 1, wherein said requesting comprises requesting the subject to identify the characteristic of the first and second sensory stimuli one of verbally, mentally, and via an input device.

8. The method of claim 1, wherein said requesting comprises requesting the subject to assign a given category to which the characteristic belongs, the given category being chosen amongst predetermined categories.

9. The method of claim 1, wherein said measuring comprises measuring the activity in a corresponding brain region responsible for integrating a sensory information related to the first and second sensory stimuli.

10. The method of claim 1, wherein said measuring comprises measuring the activity in at least one region of a frontal cortex facing a forehead of the subject.

11. The method of claim 1, wherein said measuring comprises measuring at least one of an electrical activity, a blood flow, a blood oxygenation, and a temperature.

12. The method of claim 4, wherein said applying comprises displaying a first image having the first value for the stimulus property and a second image having the second value for the stimulus property.

13. The method of claim 1, wherein said determining comprises determining a relative entropy between the first and second sensory stimuli and characterizing the subject perception of the stimulus property using the relative entropy.

14. A system for assessing a subject perception of a sensory property, comprising:
a stimulus generator for applying to a subject at least a first sensory stimulus having a first value for the stimulus property and a second sensory stimulus having a second value for the stimulus property different from the first value, the first and second sensory stimuli each comprising a characteristic to be identified by the subject during the application thereof in order to focus an attention of the subject on a decision making task, the characteristic being unrelated to the stimulus property to be assessed;
a cerebral activity sensing unit for measuring an activity of at least one brain region of the subject during the application of the first and second sensorial stimuli and the identification of the characteristic thereof in order to obtain a brain activity measurement; and
a clarity perception determining unit for determining a difference of stimulus property perceived by the subject between the first and second sensory stimuli from the brain activity measurement in order to characterize the subject perception of the stimulus property.

15. The system of claim 14, wherein the stimulus generator is adapted to display a first image having a first value for a visual clarity and a second image having a second value for the visual clarity, the first and second values for the visual clarity being set by adjusting a frequency amplitude spectrum of a given image.

16. The system of claim 14, wherein the cerebral activity sensing unit comprises one of an electroencephalography device, a functional near infrared spectroscopy device, and a functional magnetic resonance imaging device.

17. A computer-implemented method for assessing a subject perception of a stimulus property, comprising:
generating and transmitting to a stimulus generator a command indicative of at least a first sensory stimulus and a second sensory stimulus to be applied to the subject by the stimulus generator, the first sensory stimulus having a first value for the stimulus property and the second sensory stimulus having a second value for the stimulus property different from the first value, the first and second sensory stimuli each comprising a characteristic to be identified by the subject during the application thereof in order to focus an attention of the subject on a decision making task, the characteristic being unrelated to the stimulus property to be assessed;
receiving, from a cerebral activity sensing unit, a measurement of an activity of at least one brain region of the subject during the application of the first and second sensorial stimuli and the identification of the characteristic thereof; and
determining a difference of stimulus property perceived by the subject between the first and second sensory stimuli from the received measurement; and
outputting the difference of stimulus property perceived by the subject.

18. A method for identifying an adequate corrective lens for a subject, comprising:
for each one a first and a second corrective lens worn by the subject:
displaying to the subject at least a first image having a first degree of clarity and a second image having a second degree of clarity different from the first degree of clarity;
requesting the subject to identify a characteristic of the first and second images during the displaying thereof, the characteristic being unrelated to the clarity of the first and second images, thereby focusing an attention of the subject on a decision making task;
measuring an activity of at least one brain region of the subject during the displaying of the first and second images and the identification of the characteristic thereof, thereby obtaining a brain activity measurement; and
determining a difference of clarity perceived by the subject between the first and second images from the brain activity measurement, thereby obtaining the subject's perception of sensory clarity; and
identifying the adequate corrective lens as being the one of the first and second corrective lenses having the greatest difference of clarity perceived by the subject.

19. A system for identifying an adequate corrective lens for a subject, comprising:
an image generator for generating at least a first image having a first degree of clarity and a second image having a second degree of clarity different from the first degree of clarity, the first and second images each comprising a characteristic to be identified by the subject during the displaying thereof in order to focus an attention of the subject on a decision making task, the characteristic being unrelated to the clarity of the first and second images;
a display unit for displaying to the subject the first and second images received from the image generator;
a cerebral activity sensing unit for measuring an activity of at least one brain region of the subject during the displaying of the first and second images and the identification of the characteristic thereof in order to obtain a brain activity measurement; and
an adequate lens determining unit adapted to, for each one a first and a second corrective lens iteratively worn by the subject:
determine a difference of clarity perceived by the subject between the first and second images from the brain activity measurement, thereby obtaining the subject's perception of sensory clarity; and
output the difference of clarity perceived by the subject for each one of the first and second corrective lens, the adequate corrective lens being identified as being the one of the first and second corrective lenses having the greatest difference of clarity perceived by the subject.

20. A computer-implemented method for identifying an adequate corrective lens for a subject, comprising:
transmitting to a display unit at least a first image having a first degree of clarity and a second image having a second degree of clarity different from the first degree of clarity, the first and second images each comprising a characteristic to be identified by the subject during the displaying thereof in order to focus an attention of the subject on a decision making task, the characteristic being unrelated to the clarity of the first and second images;
receiving, from a cerebral activity sensing unit, a measurement of an activity of at least one brain region of the subject during the displaying of the first and second images and the identification of the characteristic; and
for each one a first and a second corrective lens iteratively worn by the subject:
determining a difference of clarity perceived by the subject between the first and second images from the brain activity measurement, thereby obtaining the subject perception of sensory clarity; and
outputting the difference of clarity perceived by the subject for each one of the first and second corrective lens, the adequate corrective lens being identified as being the one of the first and second corrective lenses having the greatest difference of clarity perceived by the subject.
